(19) United States
(12) Patent Application Publication
(10) Pub. No.: US 2025/0000441 A1
(43) Pub. Date: Jan. 2, 2025

(54) SYSTEM AND METHOD FOR EVALUATING MECHANICAL PROPERTIES OF BONE

(71) Applicant: OsteoDx Inc., Athens, OH (US)

(72) Inventor: Andrew Dick, Athens, OH (US)

(21) Appl. No.: 18/345,699

(22) Filed: Jun. 30, 2023

(51) Int. Cl. A61B 5/00

(52) U.S. Cl. CPC A61B 5/4504; A61B 5/0051; A61B 5/702

(57) ABSTRACT

A system and method are described for evaluating mechanical properties of bone tissue. One example of the system of the present invention includes a first adjustable limb constraint and a second adjustable limb constraint collectively capable of immobilizing a patient's limb, such as the patient's arm, for vibration-based, in vivo mechanical bone property testing. One example of the method of the present invention includes providing a measurement stimulus and a data acquisition system for promoting accuracy and efficiency with respect to generation, organization, and evaluation of mechanical bone property data. One example of the method of the present invention includes providing system software capable of executing a CBMT algorithm for accurately determining mechanical properties of bone tissue.

### CROSS-REFERENCE TO RELATED APPLICATIONS

The present application makes no priority claim.

### TECHNICAL FIELD

The present invention relates generally to a system and method for evaluating mechanical properties of bone tissue, and more particularly to a system and method for providing application of vibrations to bone tissue, and for permitting analysis of bone tissue responses to the applied vibrations. In one example embodiment, a patient's arm may be immobilized for vibration-based, in vivo mechanical bone property testing using a first adjustable human limb constraint, such as an elbow constraint, and a second adjustable limb constraint, such as a wrist constraint. In one example embodiment, a measurement stimulus and a data acquisition system may promote accuracy and efficient organization with respect to mechanical bone property data. In one example embodiment, system software may be configured to execute a CBMT algorithm which may permit accurate determination of mechanical properties of bone tissue.

### BACKGROUND AND SUMMARY OF THE INVENTION

Obtaining data regarding mechanical properties of bone tissue may be useful for any number of orthopedic medicine practices or related purposes. For example, it may be useful for diagnosing and treating osteoporosis (e.g., rapid loss of bone strength), evaluating and treating bone injuries (e.g., bone fractures), addressing poor bone strength and stiffness (e.g., related to prolonged disuse of one or more bones), some combination thereof, or the like. Known methods of obtaining said data include by way of example and not limitation, photon absorptiometry, dual X-ray absorptiometry (“DXA”), peripheral quantitative computed tomography (“pQCT”), and the like.

Although the aforementioned known methods have been effective to a degree at providing doctors, other medical professionals, medical researchers, and the like with some information about mechanical bone properties (e.g., DXA and pQCT may provide a 3D-representation of bone tissue), there may be significant drawbacks with each known method. With photon absorptiometry, for example, test subject (also referred to herein as “patient”) exposure to ionizing radiation may have adverse health effects on the patient. Furthermore, photon absorptiometry may do little to reveal the condition of a bone matrix itself. With DXA and pQCT, for example, although bone mineral density may be provided, other mechanical bone property information (e.g., bone stiffness) may be excluded. DXA may also require radiation and a certified operator. DXA studies may also lack reproducibility.

In recent decades, techniques for evaluating the response of bones to mechanical vibrations applied thereto have been developed for estimating mechanical properties of bone tissue (referred to herein in as “known bone vibration method(s)”). One known bone vibration method involves quasistatic mechanical testing (“QMT”). With QMT, force applied to a bone specimen may be measured and compared to bone displacement during 3-point bending tests, flexure tests, and the like. Another known bone vibration method involves applying an electromagnetic exciter or shaker to a test subject at a number of different frequency ranges, and applying an equation of motion to measured data in an attempt to distinguish between bone properties and soft tissue properties (see e.g., U.S. Pat. No. 5,006,984). Yet another known bone vibration method involves applying static and oscillatory forces over a range of frequencies to a test subject, and, from measured response data, determining functions that may be adapted to have parametric mathematical models fitted thereto (see e.g., U.S. Pat. No. 10,299,719 B2; U.S. Pat. Pub. No. 2021/0045636 A1). The parametric mathematical models may provide estimated bone stiffness.

Known bone vibration methods may demonstrate some benefits. For example, QMT may provide direct measurements of bone bending strength and stiffness. However, there are a number of drawbacks to said known methods. With QMT, excision of bone(s) and/or bone sample(s) is required for direct measuring of bone bending strength and stiffness, thus QMT has limited usefulness for in vivo testing. Furthermore, with certain known bone vibration methods (e.g., QMT applied in vivo), soft tissue proximate to the bone(s) being evaluated may mask force and displacement of the bone tissue being evaluated, which may negatively affect the accuracy of bone response data.

For applying mechanical vibrations in vivo to a patient, substantial immobilization of at least one of the patient's limbs may be required. Another significant problem with known methods and techniques for evaluating the response of bones to mechanical vibrations may include limited adjustability and/or positioning restraints with respect to test subject limb immobilization equipment. A related problem may include discomfort caused to the patient by limb immobilization equipment, and/or other equipment employed for mechanical vibration testing. Known equipment employed for mechanical vibration testing may further be more expensive than optimal, more complex (e.g., involving a large number of component parts) (e.g., difficult to install, transport, reassemble, some combination thereof, or the like) than optimal and/or difficult for doctors, technicians, researchers, or the like to operate, some combination thereof, or the like.

Yet other significant problems with known bone vibration methods may include, larger than optimal margins of error in data, inaccuracies in data, poor data organization, some combination thereof, or the like. As a specific, non-limiting example, known hardware and software for certain known bone vibration methods often suffered from a number of different errors (e.g., from application of unknown static load magnitude, from improper probe placement, some combination thereof, or the like). Certain known bone vibration methods may be susceptible to errors resulting from differences in the best model fit to the data, applying an improper frequency range when fitting a model to empirical data, inadequate criteria for determining acceptability of results, some combination thereof, or the like.

The aforementioned shortcomings speak to the need for an improved technique for measuring mechanical bone properties using mechanical vibrations.

In view of this, it is beneficial to have a system and method involving the application of mechanical vibrations to bone tissue to evaluate a response thereto, where error margins in mechanical response data may be minimized, data gathering efficiency may be streamlined, patient comfort may be maximized, and system cost(s) and complexity may be optimized.

Accordingly, an exemplary embodiment of the present invention provides a system and its corresponding method for evaluating mechanical properties of bone tissue. An exemplary system may involve no radiation, be non-invasive, may be substantially painless to a patient, and may be readily repeatable to administer to different patients.

According to the present invention in one aspect, an exemplary system for evaluating mechanical properties of bone tissue comprises an arm support comprising an adjustable wrist constraint and an adjustable elbow constraint (also referred to herein as “elbow positioner”) for immobilizing a limb of a test subject. The system may further comprise a probe, configured to apply static and oscillatory forces over a range of frequencies to the test subject. The system may further comprise a processor, configured to collect force and acceleration data from the test subject, and to determine therefrom accelerance frequency response data to determine a complex compliance frequency response function and a complex stiffness frequency response function. The processor may further be configured to determine a number of physical parameters based on a complex compliance frequency response function or a complex stiffness frequency response function. The adjustable elbow constraint may comprise a first clamp configured to be positioned proximate to a second clamp to secure an elbow proximate thereto. Each the first clamp and the second clamp may be movable along and adapted to be secured to a horizontal support member.

According to the present invention in another aspect, an exemplary system for evaluating mechanical properties of bone tissue comprises an arm support comprising an adjustable wrist constraint and an adjustable elbow constraint, a probe, and a processor. In this particular embodiment, the processor may be configured to determine a number of physical parameters based on a complex compliance frequency response function or a complex stiffness frequency response function. The processor may further be configured to execute a windowing function, the windowing function capable of causing a fitting algorithm to focus on a particular subset of data based on a frequency range of the particular subset of data. The processor may additionally be configured to determine each of the number of physical parameters by executing an algebraic function between polynomial coefficients and each of the number of physical parameters. The processor may also be configured to execute a least-squares error fitting algorithm to solve for the polynomial coefficients.

With exemplary embodiments of the present invention, mechanical properties of bone(s) may be determined based on accurate data, patient comfort may be promoted, data gathering efficiency may be promoted, and system cost(s) and complexity may be substantially optimized. Additional advantages will become apparent to those of ordinary skill in the art based on the drawings and detailed description thereof.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates multiple perspective views of a preferred cortical bone mechanics testing (“CBMT”) device, wherein a patient is present, in accordance with an exemplary embodiment of the present invention;

FIG. 2 illustrates an alternative perspective view of the preferred CBMT device of FIG. 1, wherein a patient is not present;

FIG. 3 illustrates an alternative perspective view of the device of FIG. 2;

FIG. 4 illustrates a front elevational view of another preferred CBMT device, wherein a patient is not present, in accordance with an exemplary embodiment of the present invention;

FIG. 5 illustrates preferred logic of an exemplary bone mechanics testing system of the present invention;

FIG. 6 illustrates other preferred logic of an exemplary bone mechanics testing system of the present invention;

FIG. 7 illustrates yet other preferred logic of an exemplary bone mechanics testing system of the present invention;

FIG. 8 illustrates a perspective view of an end effector and probe proximate to a patient's arm, the arm substantially immobilized by a wrist constraint and an elbow constraint, in accordance with an exemplary embodiment of the present invention;

FIG. 9 illustrates logic for a preferred modal transmission model, in accordance with an exemplary embodiment of the present invention;

FIG. 10 illustrates a CBMT algorithm, in accordance with an exemplary embodiment of the present invention;

FIG. 11 illustrates an exemplary force versus displacement curve of the present invention;

FIG. 12 illustrates another exemplary force versus displacement curve of the present invention;

FIG. 13 illustrates an exemplary accelerance versus frequency curve of the present invention;

FIG. 14 illustrates another exemplary accelerance versus frequency curve of the present invention;

FIG. 15 illustrates an exemplary compliance versus frequency curve of the present invention;

FIG. 16 illustrates another exemplary compliance versus frequency curve of the present invention;

FIG. 17 illustrates an exemplary bone stiffness versus frequency curve of the present invention;

FIG. 18 illustrates another exemplary bone stiffness versus frequency curve of the present invention;

FIG. 19 illustrates an exemplary site plot of the present invention;

FIG. 20 illustrates an exemplary compliance magnitude plot of the FIG. 19 embodiment;

FIG. 21 illustrates another exemplary compliance magnitude plot of the FIG. 19 embodiment;

FIG. 22 illustrates another exemplary site plot of the present invention;

FIG. 23 illustrates an exemplary compliance magnitude plot of the FIG. 22 embodiment;

FIG. 24 illustrates another exemplary compliance magnitude plot of the FIG. 22 embodiment;

FIG. 25 illustrates yet another exemplary site plot of the present invention;

FIG. 26 illustrates an exemplary compliance magnitude plot of the FIG. 25 embodiment;

FIG. 27 illustrates another exemplary compliance magnitude plot of the FIG. 25 embodiment;

FIG. 28 illustrates yet another exemplary site plot of the present invention;

FIG. 29 illustrates an exemplary compliance magnitude plot of the FIG. 28 embodiment;

FIG. 30 illustrates another exemplary compliance magnitude plot of the FIG. 28 embodiment;

FIG. 31 illustrates exemplary logic of an exemplary embodiment of the present invention;

FIG. 32 illustrates exemplary mean bone loss data in accordance with an exemplary embodiment of the present invention;

FIG. 33 illustrates exemplary percent change in bone data in accordance with an exemplary embodiment of the present invention;

FIG. 34 illustrates a left-side elevational view of another exemplary CBMT device of the present invention;

FIG. 35 illustrates a perspective view of the exemplary CBMT device of FIG. 34;

FIG. 36 illustrates a perspective view of the exemplary CBMT device of FIG. 34 having a first and second display;

FIG. 37 illustrates a front elevational view of the exemplary CBMT device of FIG. 36;

FIG. 38 illustrates a perspective view of an exemplary adjustable wrist constraint of the exemplary CBMT device of FIG. 34;

FIG. 39 illustrates a right-side elevational view of the exemplary adjustable wrist constraint of FIG. 38;

FIG. 40 illustrates a front elevational view of the exemplary adjustable wrist constraint of FIG. 38;

FIG. 41 illustrates a rear elevational view of the exemplary adjustable wrist constraint of FIG. 38;

FIG. 42 illustrates a top plan view of an exemplary elbow positioner of the exemplary CBMT device of FIG. 34;

FIG. 43 illustrates a perspective view of the exemplary elbow positioner of FIG. 42;

FIG. 44 illustrates a front elevational view of the exemplary elbow positioner of FIG. 42;

FIG. 45 illustrates a top plan view of an exemplary shaker assembly isolation system of the exemplary CBMT device of FIG. 34;

FIG. 46 illustrates a perspective view of the exemplary shaker assembly isolation system of FIG. 45;

FIG. 47 illustrates a front elevational view of the exemplary shaker assembly isolation system of FIG. 45;

FIG. 48 illustrates a perspective view of the exemplary shaker assembly of the exemplary shaker assembly isolation system of FIG. 45;

FIG. 49 illustrates another perspective view of the exemplary shaker assembly of FIG. 48;

FIG. 50 illustrates a front elevational view of the exemplary shaker assembly of FIG. 48;

FIG. 51 illustrates a bottom plan view of the exemplary shaker assembly of FIG. 48; and

FIG. 52 illustrates a front elevational view, a top plan view, and two exploded perspective views of an exemplary linear guide bearing system of the exemplary shaker assembly isolation system of FIG. 45.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein may be made without departing from the scope and spirit of the present invention. In addition, descriptions of well-known functions and constructions are omitted for clarity and conciseness.

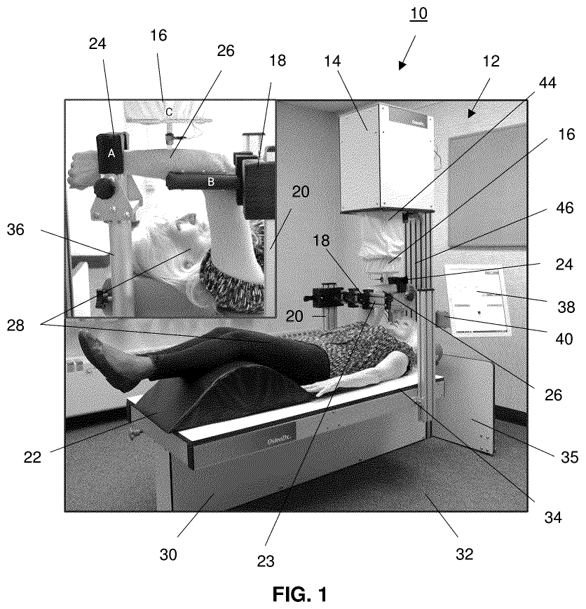

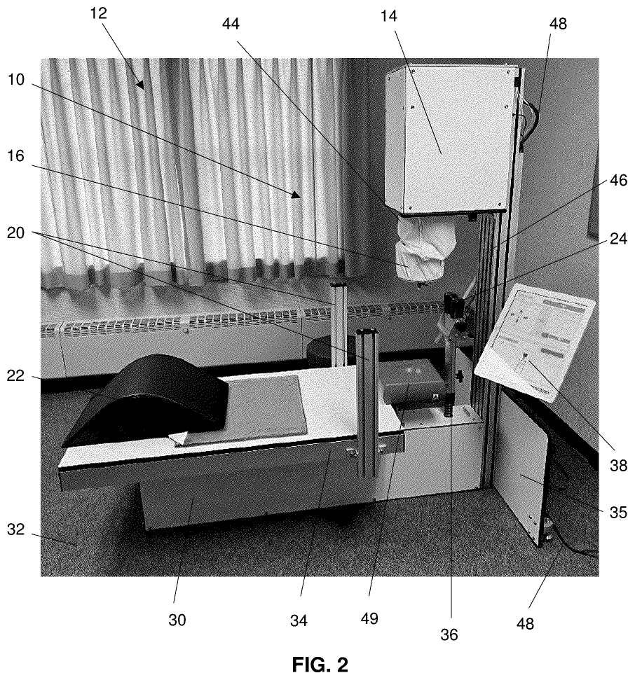

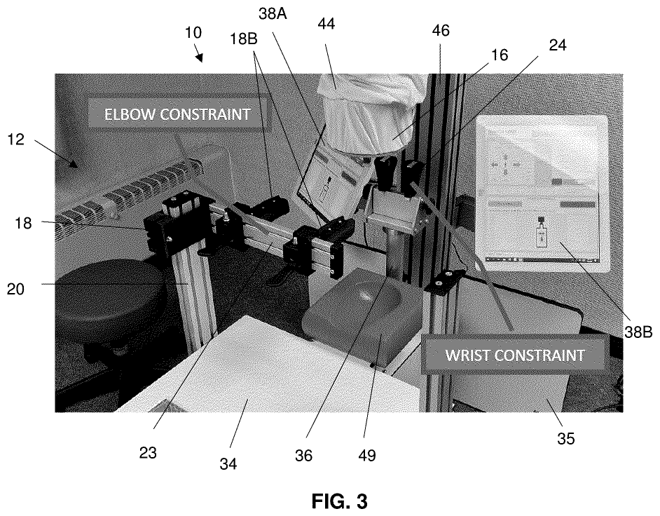

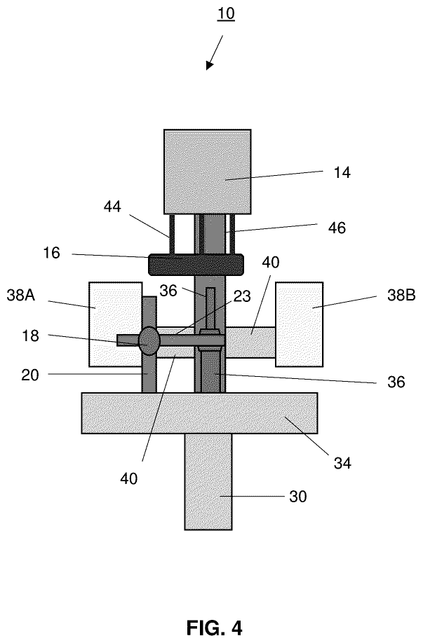

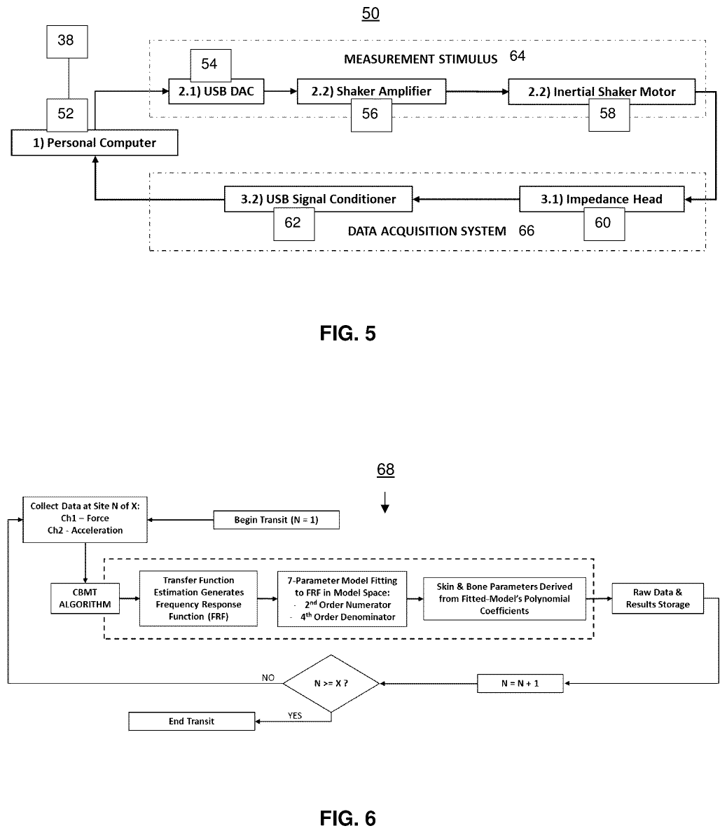

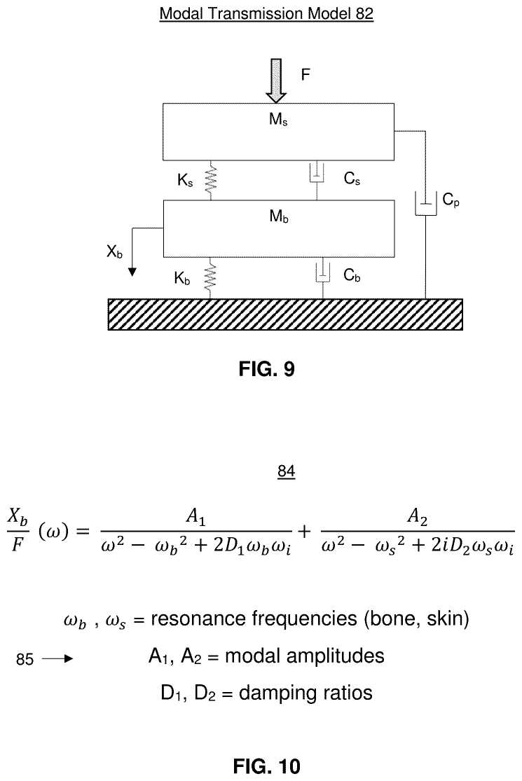

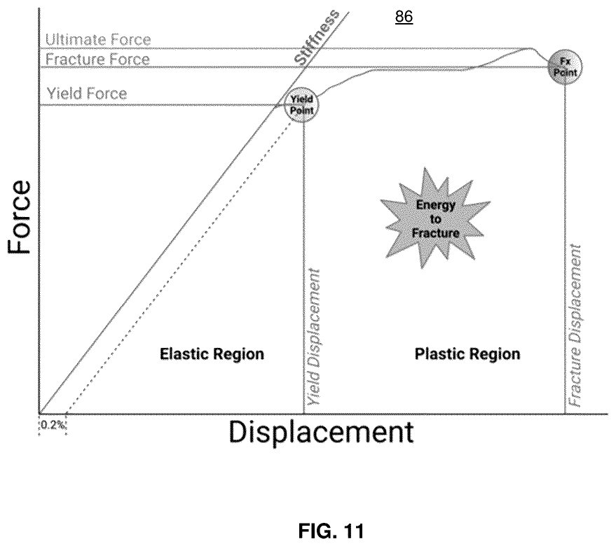

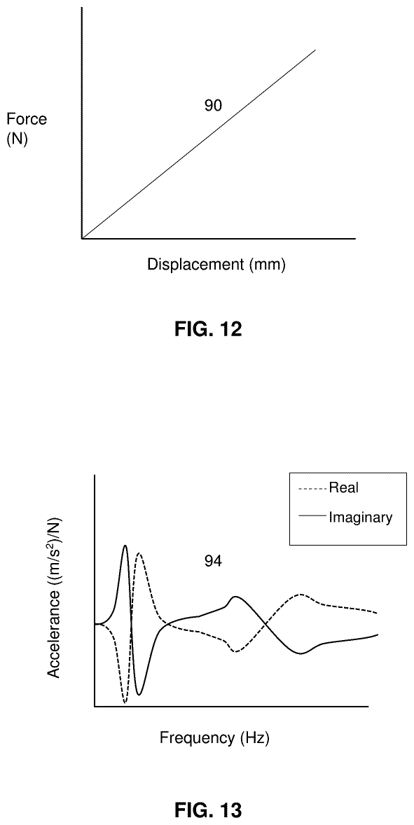

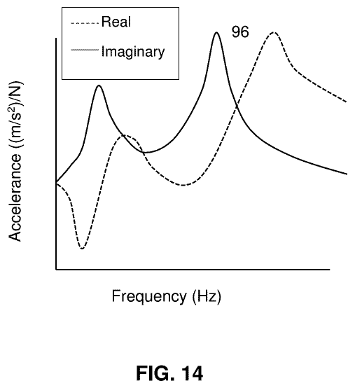

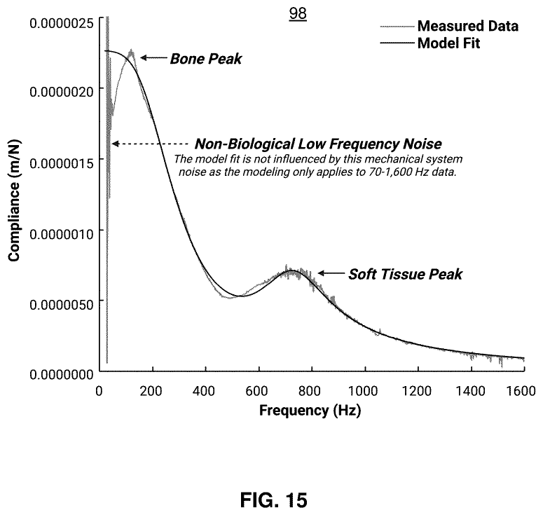

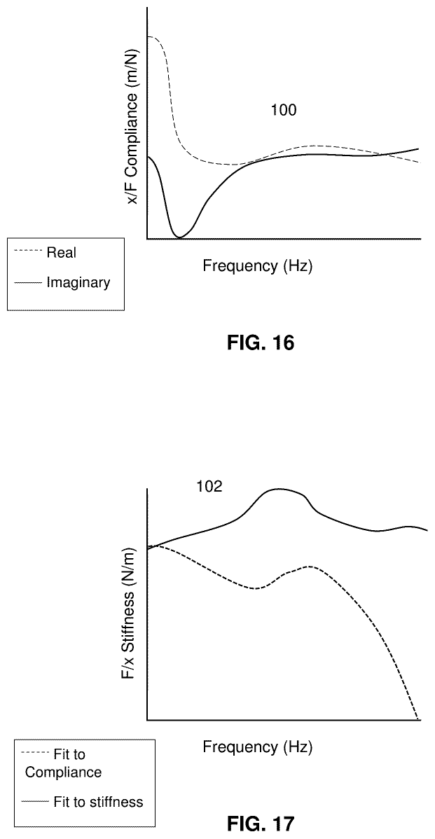

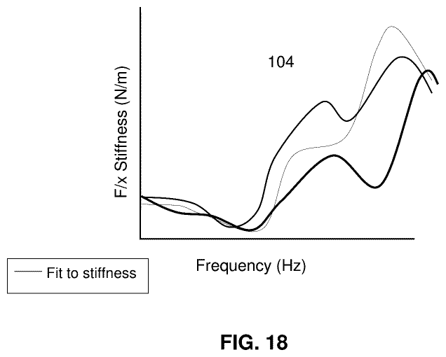

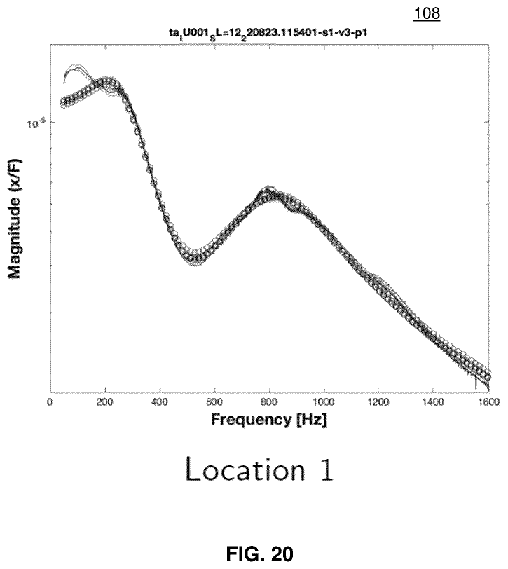

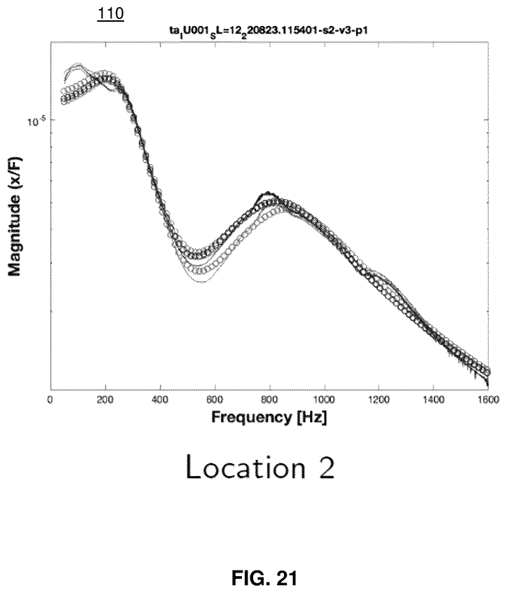

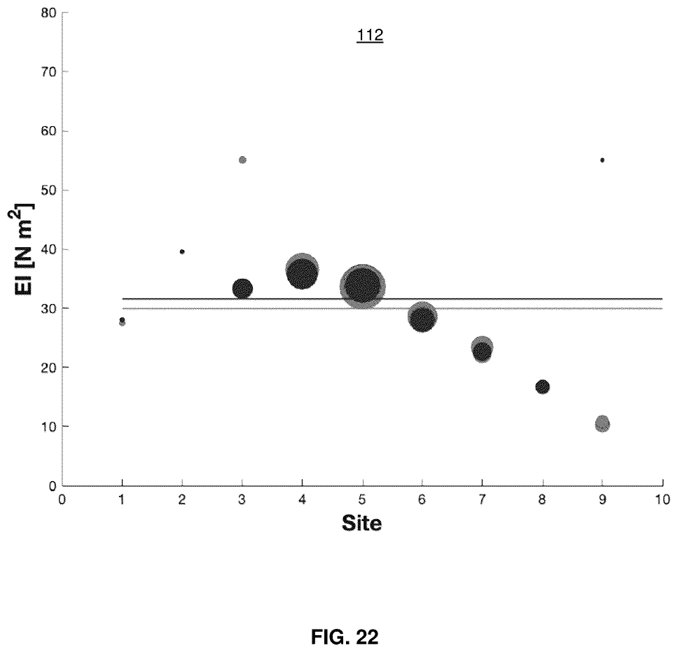

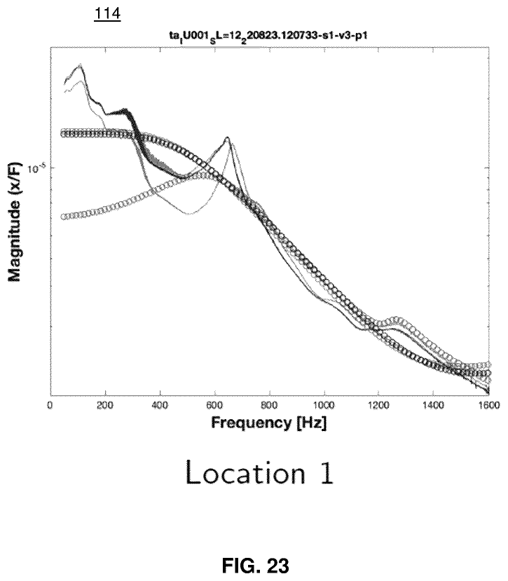

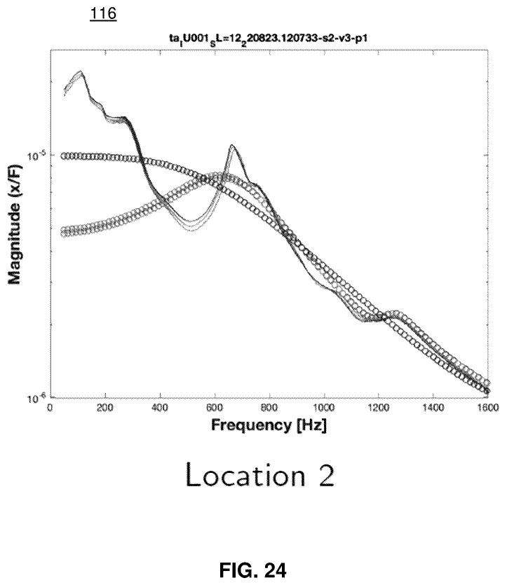

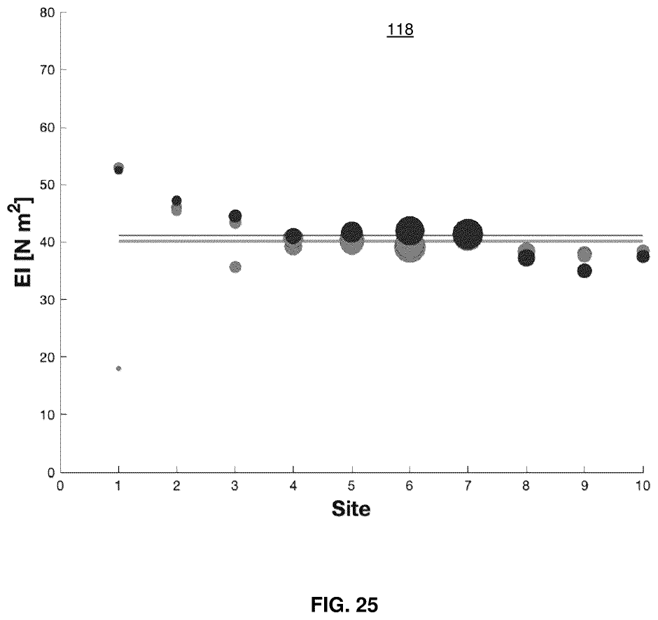

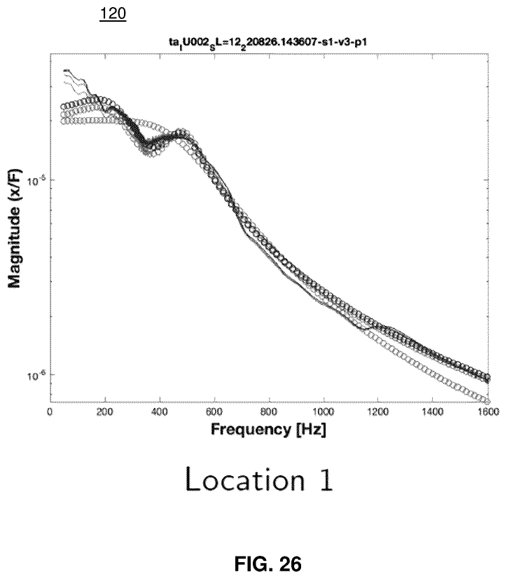

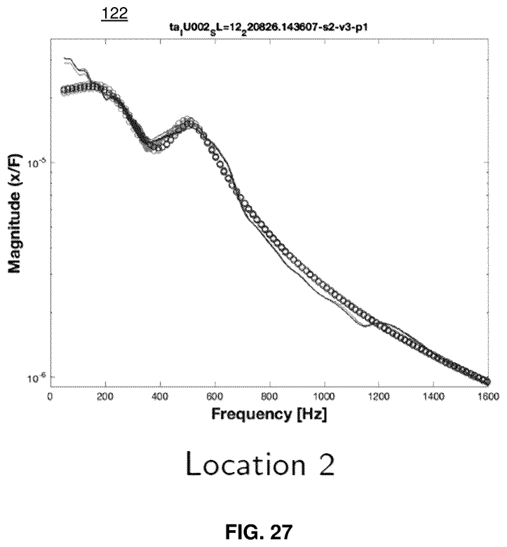

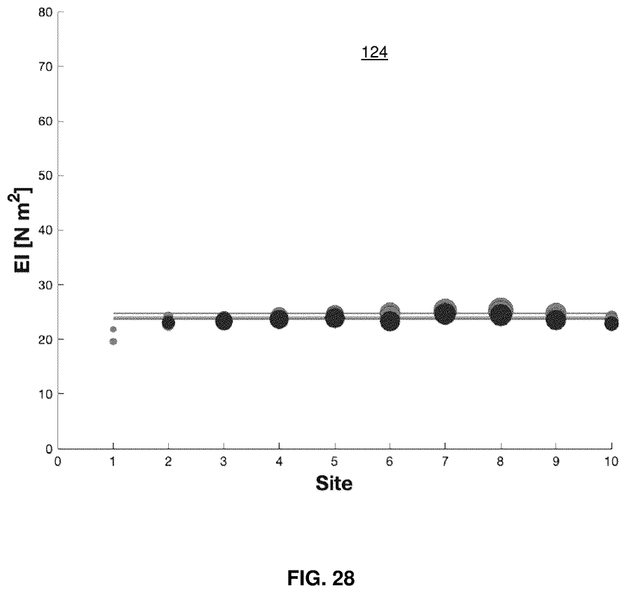

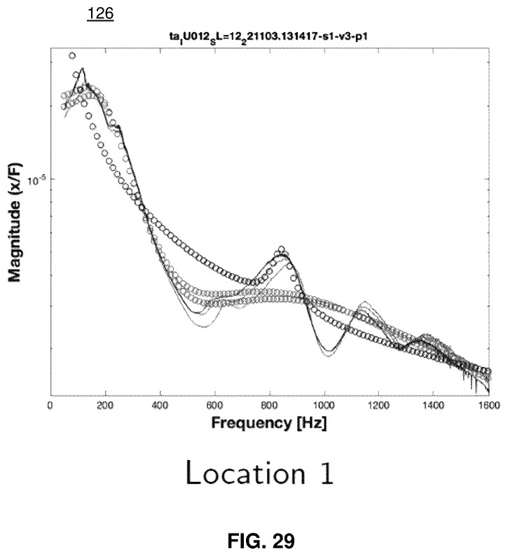

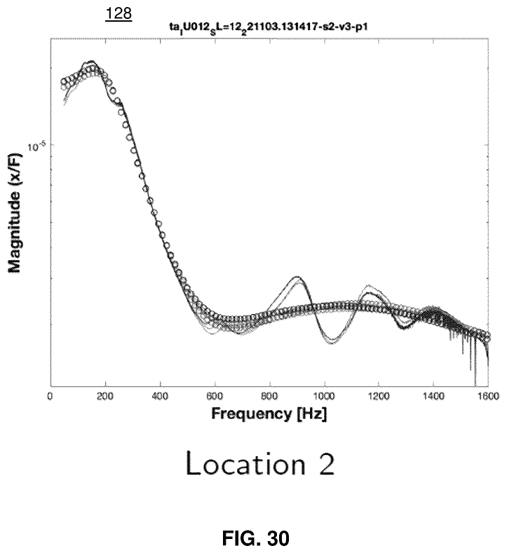

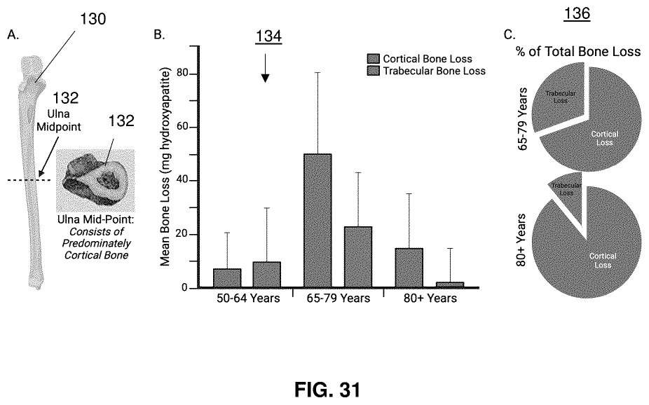

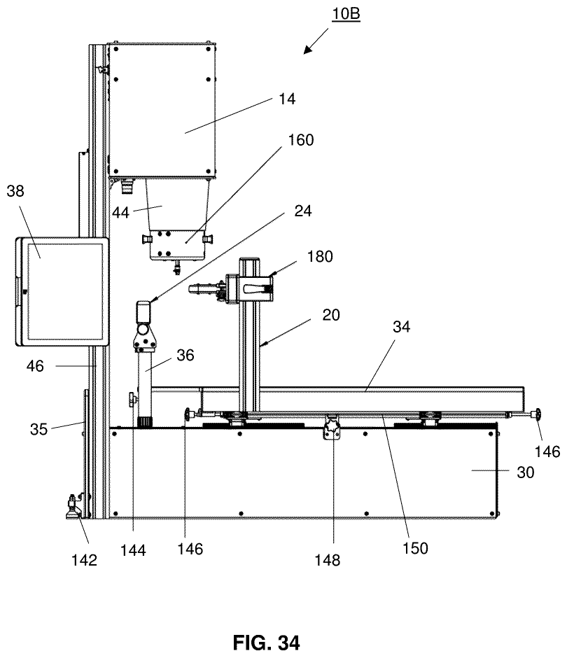

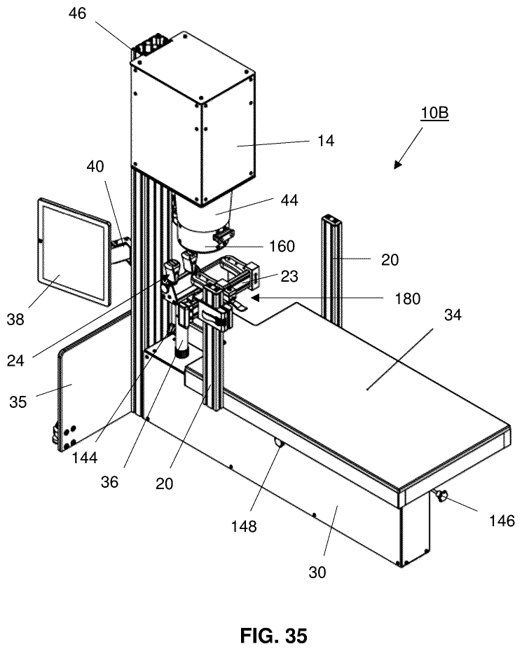

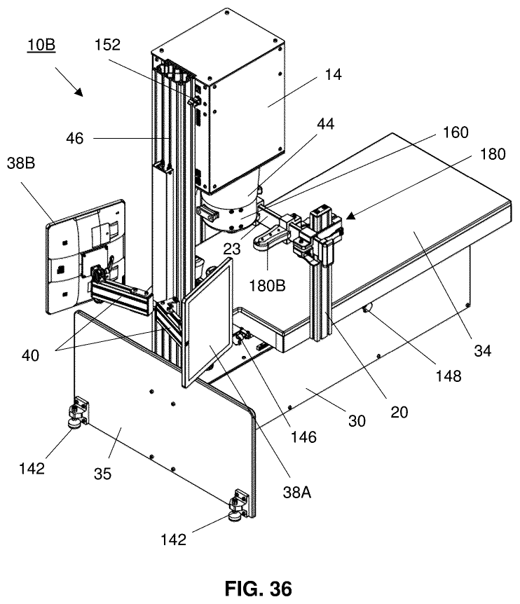

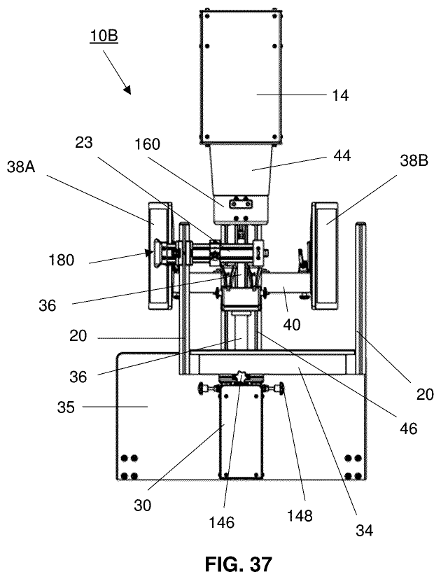

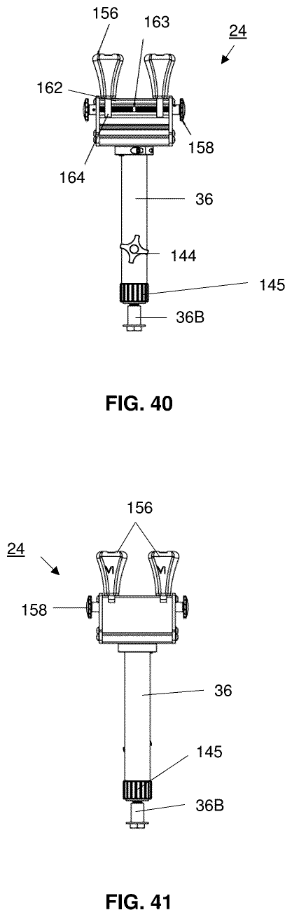

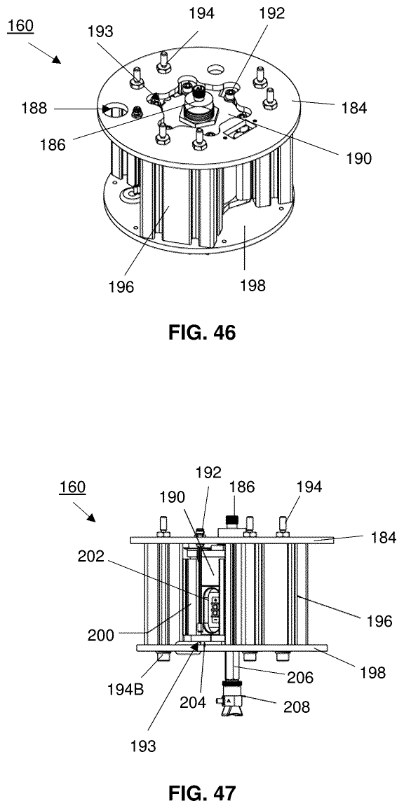

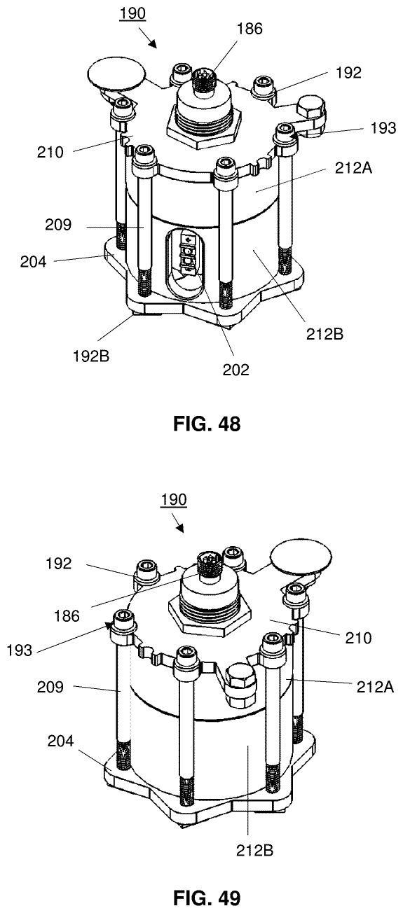

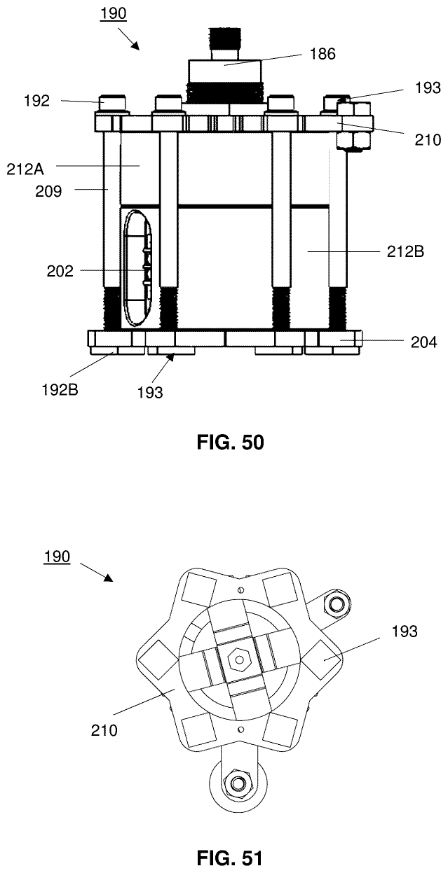

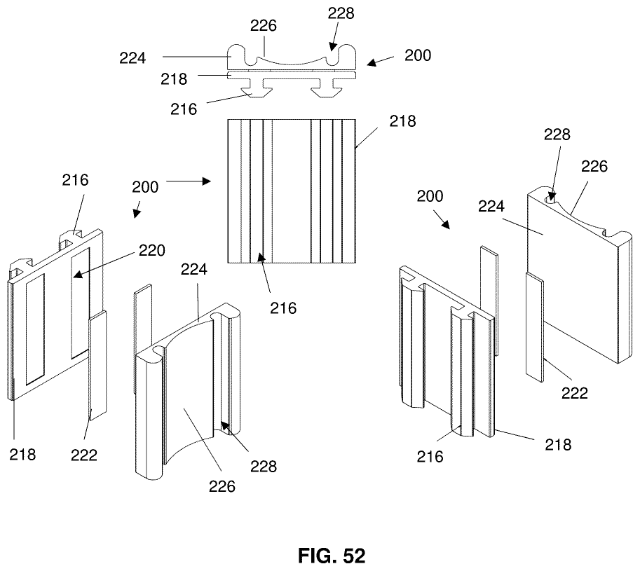

FIGS. 1-4 and 8 generally illustrate various views of an exemplary CBMT device of the present invention and components thereof, FIGS. 34-52 generally illustrate various views of another exemplary CBMT device of the present invention and/or components thereof, FIGS. 5-7 generally illustrate exemplary logic of a preferred embodiment of the present invention, FIGS. 9-10 generally illustrate an instance of a lumped-parameter model representing a skin-bone complex, FIGS. 11-30 generally illustrate exemplary data generation, organization, evaluation, validation, some combination thereof, or the like of certain preferred embodiments, and FIGS. 31-33 generally illustrate exemplary mechanics of bone loss due to aging.

Referring initially to FIGS. 1-4, an exemplary CBMT device 10 (in medical facility room 12) may involve any number of different component parts, and may be adapted to be assembled, mobilized on wheels, broken down and reassembled, transported, some combination thereof, or the like without departing from the scope of the present invention. Exemplary CBMT may be beneficial for evaluating bone geometry, tissue material properties and composition, bone porosity, microarchitecture, nanoscale collagen cross-linking, protein mineral bonding, some combination thereof, or the like. It will be apparent to one of ordinary skill in the art that there may be any number of different applications of CBMT employed without necessarily departing from the scope of the present invention.

A patient 28 may need evaluation of one's mechanical bone properties for any number of different reasons, including by way of example and not limitation, diagnosis of a certain bone disease (e.g., osteoporosis), injury, deficiency, deformity, some combination thereof, or the like (referred to herein as “bone issue”), monitoring of improvements (or lack thereof) of a bone issue over time, some combination thereof, or the like. To implement testing of mechanical bone properties in accordance with an exemplary system, the patient 28 may be positioned on table 34. The table 34 may be supported above a floor 32 by an exemplary blade or support apparatus 30. The table 34 may comprise a rigid and stable platform. The table 34 may be configured to be adjusted in the left to right directions, head to toe directions, some combination thereof, or the like to promote positioning of a patient's limb to be tested, such as the patient's 28 arm 26. The patient's 28 legs may be positioned above an elevation device 22 to promote comfort of the patient 28. A head rest 49 may further be provided to permit patient 28 comfort.

In certain embodiments, an exemplary CBMT device 10 may comprise multiple monitors comprising two or more displays (e.g., 38A-B) proximate to the CBMT device, but such is not required. Each display (e.g., 38A-B) may be mechanically connected 40 to a vertical post 46, although such is not required. The vertical post 46, support apparatus 30, and a horizontal support member 35 may collectively provide structural support for the device 10. The display 38 may be configured to permit a doctor, technician, researcher, or the like to view, configure, adjust, monitor, and otherwise engage with certain aspects of software of an exemplary system for evaluating mechanical properties of bone. Said engagement may occur by way of one or more interfaces, which may include one or more configurable areas. The display 38 may permit a doctor to monitor cortical bone properties in real time as mechanical vibrations are being applied to the patient's limb. Aspects of an exemplary system may be implemented according to one or more exemplary software modules. Exemplary software instructions may be executed by one or more processors. Information may be communicated to system users and/or administrators by way of any number of different computer readable mediums.

Software for an exemplary system and its corresponding method may be implemented using MATLAB, JAVA, CGI script, Python, some combination thereof, or the like. It will be apparent to one of ordinary skill in the art that any number of different computing devices may be employed to execute exemplary software of the present invention. Computing devices, preferably adapted to run programming code and implement various instructions and/or functions of software of an exemplary system and its corresponding method, may include processors, microprocessors, microcontrollers, embedded processors, DSP, some combination thereof, or the like. Exemplary software may be stored on an electronic storage medium, and executed with the cooperation of a controller and memory.

Although the exemplary device 10 of the FIGS. 1-4 embodiment may comprise one or more displays 38 proximate to the device 10, such is not necessarily required with other embodiments. Any number of different computing and/or display devices, whether or not employed proximate to an exemplary CBMT device, may be employed without departing from the scope of the present invention. Said devices may include by way of example and not limitation, smart phones, other smart devices, desktop computers, tablets, some combination thereof, or the like, each of which may be employed to both execute and display aspects of an exemplary embodiment.

An exemplary software display device may include a screen, a configurable area thereof, gesture capture regions, any number of screen sensors, a camera, some combination thereof, or the like. An exemplary computer device, adapted to permit a doctor, researcher, technician, or the like to interact with software of an exemplary system, may be in electronic communication with a system database adapted to store data from any number of different users. One or more control computing devices may be adapted to regulate multiple CBMT devices and/or data thereof, but such is not necessarily required. It will be apparent to one of ordinary skill in the art that there may be any number of different devices or methods available permitting doctors, researchers, technicians, or the like to interact with software of an exemplary system without departing from the scope of the present invention.

In an exemplary embodiment, a doctor, researcher, technician or the like may only interact with the system after entering an approved username and password. User access and user roles may be defined based on any number of different methods to regulate the level of access certain users have to software of an exemplary system.

An exemplary CBMT device 10 may be configured to utilize vibration analysis to measure ulna bone transverse bending stiffness (the ulna may be ideally suited for a bending test due to, e.g., high cortical bone presence at a midpoint thereof). An exemplary transducer may be attached or positioned proximate to an end-effector 16 for transmitting tissue force and acceleration response signals to one or more processors. The end-effector 16 may be adjusted in any number of different directions in three dimensions, based on, by way of example and not limitation, a positioning apparatus 44 (e.g., 3-motor standard delta-robot motor configuration). It is known a strong correlation exits between bone stiffness and bone strength, and any number of doctors, technicians, researchers or the like may seek to determine bone stiffness (based on an exemplary embodiment) for any number of different patients (e.g., to address any number of different bone-related health issues) accordingly. Motors, computers, other electrical equipment, some combination thereof, or the like may be housed in a control cabinet 14, which may be positioned above the vertical post 46.

Still referring to FIGS. 1-4, in this embodiment of the invention, the patient's 28 arm 26 may be immobilized by an adjustable elbow constraint 18 and an adjustable wrist constraint 24. The adjustable elbow constraint 18 may comprise a block pair apparatus comprising a number of blocks, the block pair apparatus adjustable along a height of a vertical support 20 (also referred to herein as “vertical frame member 20”) (e.g., to accommodate any number of arm sizes, lengths, positions, some combination thereof, or the like for any number of different tests). One or more grooves of each vertical support 20 may be configured to receive one or more protrusions of each block of the block pair apparatus. A first block of the block pair apparatus may be positioned opposite of a second block of the block pair apparatus, and the first block may be mechanically connected to the second by, for example not by way of limitation, an adjustable bolt, fastener, clip, some combination thereof, or the like.

The mechanical connection may be adjusted to cause the first block and the second block to be positioned in close enough proximity to one another to secure the block pair apparatus to the vertical support 20. The mechanical connection may also be adjusted to cause the first block and the second block to be positioned away from one another enough to permit adjustment of the height of the block pair apparatus with respect to the vertical support 20 (e.g., to accommodate different arm lengths, elbow positions, some combination thereof, or the like for various testing), and/or to permit removal of the block pair apparatus from the vertical support 20 (e.g., to permit switching the block pair apparatus from one vertical support 20 to another vertical support 20 to permit testing of either a left arm or a right arm).

The adjustable elbow constraint 18 may further comprise a number of clamps 18B specifically configured to secure an elbow of an arm 26 proximate to a horizontal support 23 (also referred to herein as “horizontal frame member 23”). Each clamp may comprise an amount of cushion material positioned to contact the arm 26 to promote both immobilization of the arm 26 and patient comfort during testing. The arm may be positioned substantially perpendicular to a test probe.

Each clamp 18B may include a number of protrusions each adapted to be received by a corresponding groove of the horizontal support 23 to permit each clamp 18B to be repositioned along a length of the horizontal support 23. Each clamp 18B may be secured to the horizontal support 23 by tightening an adjustment feature of the clamp 18B (e.g., a bolt, clip, fastener, some combination thereof, or the like) to affix a surface of the clamp 18B to a surface of the horizontal support 23. One clamp may be positioned a particular distance from the other clamp to secure an elbow therebetween. The horizontal support 23 may be mechanically connected to the block pair apparatus. Thus, adjustment of the height of the block pair apparatus may provide adjustment of the height of the horizontal support 23 (e.g., to accommodate different arm lengths, elbow positions, some combination thereof, or the like for various testing).

It will be apparent to one of ordinary skill in the art that the particular distance of one secured clamp with respect to another secured clamp may be varied to permit securement of any number of different elbow shapes and/or sizes at any number of different positions. It will also be apparent to one of ordinary skill in the art that the particular shapes, sizes, and materials used for exemplary adjustable elbow constraint features may be varied without departing from the scope of the present invention.

For certain exemplary tests, the shoulder rotation angle of a patient 28 may be substantially fixed at approximately 90° (humerus of arm 26 relative to table 34 surface). An exemplary embodiment (e.g., 10) may promote both rigid constraint of an arm 26 (including ulna and radius thereof) and patient 28 comfort. Each clamp 18B may include an articulating grip feature (e.g., labeled as “B” in FIG. 1) protruding from the clamp 18B, wherein the articulating grip feature may be configured to be positioned underneath epicondyles of a humerus, although such is not required. Pressure may be applied from multiple articulating grip features on opposing sides of the arm 26 to substantially immobilize the arm 26. The articulating grip features (e.g., “B” in FIG. 1), along with the adjustable table 34, may be employed to provide adjustment of an arm 26 position before and/or during testing to permit an exemplary system to determine optimal arm position for data collection (e.g., optimal positioning of the ulna, which may be achieved by aligning a humerus vertically with respect to shoulder rotation, and by aligning a longitudinal axis of the ulna with a long edge of the table 34). Pressure of the articulating grip features applied to an underside of the arm 26 (e.g., underside of humeral epicondyles) may cause the arm/forearm 26 to be suspended above the table 34 away from the rest of the patient's 28 body.

An adjustable wrist constraint 24 may comprise an adjustable clamping device (e.g., labeled as “A” in FIG. 1), a vertical position of which may be adjusted along a length of a vertical support 36. Opposing clamps of the adjustable clamping device may be positioned any number of distances from one another by, for example not by way of limitation, bolt adjustment, clip adjustment, fastener adjustment, some combination thereof, or the like. A platform (e.g., comprising aluminum) may be provided below the opposing clamps, e.g., for stabilizing the wrist there above. The clamps may be contoured and/or curved to restrict wrist rotation, accommodate different wrist shapes and/or sizes, some combination thereof, or the like.

In the particular embodiment shown, a dual lead screw is employed for permitting wrist constraint 24 repositioning (e.g., a clamping action of the constraint 24 may be achieved by turning lobed knobs). One opposing clamp may be positioned sufficiently close enough to the other opposing clamp to secure a patient's 28 wrist therebetween. Here, curved vise blocks may both capture a top part of the wrist and apply downward pressure to immobilize the wrist. Each opposing clamp may comprise an amount of cushioning material to, e.g., promote user comfort. The adjustable clamping device may be rotated about the top of the vertical support 36 to, for example not by way of limitation, promote user comfort, achieve a desired arm 26 position, some combination thereof, or the like. It will be apparent to one of ordinary skill in the art that an exemplary adjustable wrist constraint may permit substantial securement of a wrist at any number of different positions across three dimensions.

Providing specific wrist positioning (e.g., using exemplary adjustable constraints 18, 24) may be important to obtaining information about effects of ulnar positioning under a probe (positioned proximate to end effector 16). An exemplary adjustable wrist constraint 24 may permit an amount of rotation ability of the wrist (e.g., rotation between pronation and supination positions), but such is not necessarily required. In certain embodiments, wrist rotation may be substantially restricted to substantially prevent ulna repositioning (which may result in measurement errors). It will be apparent to one of ordinary skill in the art that the particular shapes, sizes, arrangements, and materials used for adjustable wrist constraint features may be varied without departing from the scope of the present invention. It will also be apparent to one of ordinary skill in the art that exemplary elbow and/or wrist positioning is not necessarily limited to the in vivo testing illustrated and described herein, and any number of living and/or cadaveric tissues may be tested using exemplary elbow and/or wrist positioning without departing from the scope of the present invention.

The application of the probe (proximate to end effector 16) to the patient's 28 arm 26 together with immobilization of the patient's 28 arm 26 by way of each an adjustable wrist constraint 24 and an adjustable elbow constraint 18 may be referred to herein as “non-invasive, 3-point bending.” The probe (proximate to end effector 16) may permit direct, functional measurement of the mechanical properties of cortical bone, including by way of example and not limitation ulna cortical bone mass, stiffness, damping, flexural rigidity, quality factor, strength, some combination thereof, or the like. Any electronic component of an exemplary CBMT device 10 may be electronically wired 48 to one or more power sources, although such is not necessarily required (e.g., certain components may alternatively or additionally be battery powered).

Referring now to FIG. 5, exemplary logic 50 for a preferred data acquisition system 66 and measurement stimulus 64 is shown. The exemplary data acquisition system (DAQ) 66 may be executed by any number of different computing devices (e.g., personal computer 52). The personal computer 52 implemented to execute the data acquisition system 66 may include standard hardware and/or a standard operating system (e.g., Windows 10), but such is not necessarily required. The personal computer may include a digital display 38, or may otherwise be linked thereto. Software instructions for an exemplary data acquisition system 66 may be implemented from MATLAB, but such is not necessarily required.

An exemplary measurement stimulus 64 for generating mechanical vibrations (e.g., in order to transduce bone measurement data) may comprise a USB sound card (e.g., 24-Bit DAC 54), a miniature inertial shaker system (including a motor 58 thereof), a shaker amplifier 56, some combination thereof, or the like. The data acquisition system 66 may comprise and impedance head 60 (e.g., dual channel impedance head for measuring force and acceleration of tissue resulting from mechanical vibrations applied thereto), a USB signal conditioner 62, an output device (e.g., 24-Bit ADC Output to PC) (e.g., certain input recognized by PC as audio signals), some combination thereof, or the like. Functions of an exemplary system may be initiated and regulated, and reactions thereof may be recorded by exemplary software (e.g., implemented from MATLAB).

Referring now to exemplary logic 68 in FIG. 6, force and acceleration of tissue data may be collected at any number of different of locations spanning a surface of a patient's body part(s) (e.g., ulna) to, for example, permit finding a position most closely aligning with a centroid. Extraneous modes of vibration may be least excited, and anterior-posterior bending may be most prevalent when an exemplary probe is positioned at a centroid (referred to herein as “dominant mode of vibration”). Furthermore, at a centroid region of a bone (e.g., ulna), the bone may consist predominately of cortical bone (most bone loss may be cortical bone for elderly people). Cortical bone may be more associated with increased incidence of fragility fractures.

A 7-parameter model, described in more detail below, may be used to express a dominant mode of vibration. Data reflecting tissue force and acceleration may be refined according to an exemplary CBMT algorithm. It will be apparent to one of ordinary skill in the art that an exemplary CBMT algorithm may be executed in real time as input from the probe is transduced, performed after input from the probe is transduced, some combination thereof, or the like without departing from the scope of the present invention.

An exemplary CBMT algorithm may involve transfer function estimation for generating a frequency response function (FRF) as described in more detail below. An exemplary CBMT algorithm may further involve 7-parameter model fitting as described in more detail below. Skin and bone parameters may be derived from polynomial coefficients of the aforementioned model. Validation of raw data may be executed, and data results may thereafter be stored to one or more databases of one or more processors of an exemplary system. It will be apparent to one of ordinary skill in the art that exemplary CMBT algorithms and related data refining techniques specifically described herein are merely illustrative, and are not exhaustive of the scope of the present invention.

A preferred CBMT device may cause repositioning of a data collection probe (which may be adjusted according to positioning of an end effector) until determination of a centroid position occurs, although such is not necessarily required. An exemplary system may index data across a bone (e.g., ulna) a specified number of times (e.g., specified by a system administrator) to ensure data is collected for a number of different locations. In certain exemplary embodiments, data analysis by the system does not necessarily lead to halting data collection at a site with calculated 7-parameters, which may be degraded from previous locations. Collected data may be stored to a disk or other storage medium before analysis of the collected data occurs (e.g., to allow the patient to leave a testing area immediately after data collection occurs), but such is not necessarily required.

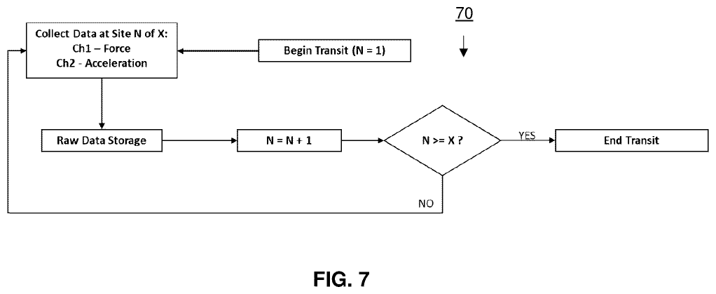

Referring now to exemplary logic 68, 70 in FIGS. 6-7, a parameterized model equation may be solved for indirectly to, e.g., determine information about mechanical properties of bone (e.g., bone stiffness). An exemplary algorithm for determining mechanical properties of bone in accordance with an exemplary embodiment may involve a data collection probe being moved to an initial position proximate to a patient's bone (e.g., on skin proximate to an ulna and surrounding tissue thereof). Exemplary data collection may involve exemplary static load application, oscillatory stimulus force signal superimposition, raw force and acceleration data signal collection, and raw data signal storage. In the FIGS. 6-7 embodiment, following the data collection probe being moved to an assigned position, a static load may then be applied to the patient's arm (or another limb). An oscillatory stimulus force signal may be superimposed to the static load. Raw force and acceleration data signals may then be collected in a time domain by way of, as a non-limiting example, a USB connected ADC signal conditioner (e.g., two-channel in line signal conditioner). Raw data signals may be stored to a disk or other digital storage medium.

Thereafter, the data collection probe may be assigned to a separate, subsequent lateral position on the patient's arm, and exemplary data collection may be repeated in accordance with the FIGS. 6-7 embodiment. Reassignment and subsequent data collection may occur for the specified number of data collection sites. Conformity measures with respect to the response data may improve upon each lateral shift with respect to application of static and oscillatory forces from an exemplary probe. An optimum location for data collection may be realized where a measure of conformity is maximized.

After data collection occurs for all specified data collection sites, data analysis (e.g., in accordance with an exemplary CBMT algorithm) may occur, wherein data analysis may involve a parameterized model. A parameterized mathematical model may account for the mass of the skin (Mₛ), transverse bending stiffness of the skin (Kₛ), damping coefficient of the skin (Cₛ), mass of the bone (M_{b}), transverse bending stiffness of the bone (K_{b}), damping coefficient of the bone (C_{b}), and damping coefficient of peripheral flesh around the bone (Cₚ). The present invention may involve any number of different parametric mathematical models, and the aforementioned model is in no way exhaustive of the scope of the present invention.

In an exemplary embodiment, raw force input data and acceleration output data comprise the time domain signals (functions may be expressed as F(t) and a(t), respectively), and may be used to generate a complex accelerance (m/s2/N) frequency (Hz) response function (also referred to herein as “aFRF”). An exemplary ADC signal conditioner may be employed for collecting raw force and acceleration data signals.

F(t) (applied force over time) and a(t) (tissue acceleration measured over time) may be expressed as functions of frequency using well known mathematical methods, and combined into an accelerance function (acceleration per unit force) using well known mathematical methods. Complex notation may be used to express magnitude and phase information of accelerance compared to frequency. Alternatively, or additionally, viewing separate real and imaginary complex data in a frequency domain may be utilized. An exemplary aFRF may be integrated twice to generate a complex compliance frequency response function (also referred to herein as “cFRF”) with units (m/N). An exemplary cFRF may be inverted to express a complex stiffness frequency response function (also referred to herein as “sFRF”) with units (N/m). An exemplary sFRF may demonstrate bone stiffness as a function of vibration frequency.

A sub-range of the total frequency spectrum of interest may comprise work in model space with the cFRF data. Solutions to coefficients of the transfer function polynomial may be solved through a least-squares error fitting algorithm. Based on algebraic relationships between polynomial coefficients and the 7 aforementioned physical model parameters, solutions may be generated for the 7 physical model parameters, and said solution may be stored to a disk or other digital storage medium. An exemplary measure of conformity may include the differences between each cFRF and sFRF. Data for each of the aforementioned 7-parameters may include derived 7-parameters for both cFRF and sFRF model fits. The aforementioned data may be stored to a disk or other digital storage medium. The aforementioned work in the model space, 7 physical model parameter determination, and data storage may be repeated for sFRF data. It will also be apparent to one of ordinary skill in the art the 7 parameters described herein are merely illustrative, and any number of different parameters may be employed without necessarily departing from the scope of the present invention.

The aforementioned work in the model space, 7 physical model parameter determination, and data storage may be repeated for a new sub-range of total frequency spectrum of interest. Any number of different sub-ranges of total frequency spectrum of interest may be tested until all relevant sub-frequency ranges have been calculated. Thereafter, solution selection may involve determining the percentage Root-Mean-Square between the 7-parameters of cFRF and sFRF (referred to herein as “% RMS7P”) for each sub-frequency range (e.g., between 20 Hz and 1600 Hz). The % RMS7P smallest in magnitude may dictate the set of 7-parameters to be selected as the best model fit to the transfer function, and may be used to make determinations about a patient accordingly. The aforementioned exemplary data analysis may be repeated for each lateral probe position until % RMS7P begins to degrade. The 7-parameters (e.g., K_{b}) calculated at a site immediately prior to degradation of % RMS7P may be the preferred parameters used for evaluating mechanical properties of the bone tested.

Exemplary parameter determinations based on sFRF data illustrated and described herein may demonstrate a higher degree of data accuracy compared to known parameter fitting techniques. As opposed to determining a best EI, exemplary parameter determinations involve determining the lowest mean squared error (MSE) between data and model fits as described above.

A windowing function may be employed to specify where parameter fitting efforts are to be directed. An exemplary windowing function may be beneficial for emphasizing specific regions of interest for data analysis (e.g., as opposed to using a time-consuming hunting algorithm to sift through hundreds or more sub-regions of interest to find an optimized data set). With an exemplary windowing function, frequency ranges of interest may be assigned a varying window weighting factor to cause a fitting algorithm to focus on a particular subset of data. By way of example and not limitation, the 7-parameter may be directed by an exemplary windowing function to accurately fit sFRF or cFRF data in a lower frequency sub-region of applied vibrations, said sub-region encompassing a primary mode of vibrations relevant to mechanical bone characteristics (referred to herein as “bone mode”). The aforementioned model may further be directed by an exemplary windowing function to fit sFRF data in a higher frequency sub-region of applied vibrations (accuracy of fitting may be less critical compared to lower frequency sub-region fitting), said sub-region encompassing a secondary mode of vibrations relevant to soft tissue characteristics (referred to herein as “skin mode”). An entire frequency range stimulated by the control signal may then be analyzed. Frequency may range between 20 Hz and 1600 Hz, although it will be apparent to one of ordinary skill in the art that any number of different frequencies may be tested without necessarily departing from the scope of the present invention. The waveform of mechanical vibration frequencies introduced may be chirp waveform.

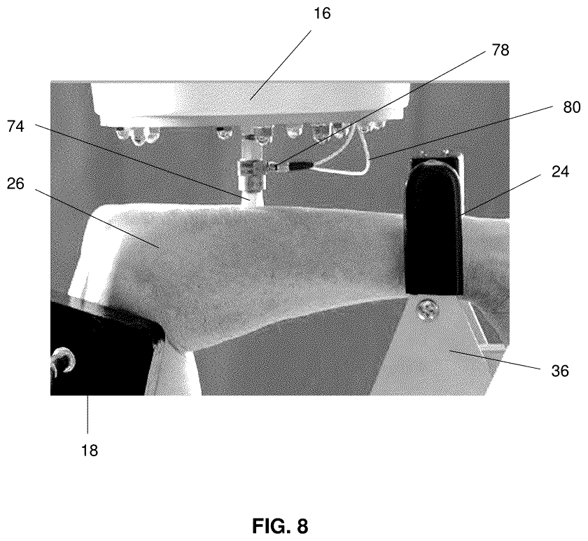

Referring now to FIG. 8, a data acquisition probe 74 may be mounted to the underside of an end-effector 16, and may be in mechanical communication 78 with a shaker motor (not shown) therein, and/or in electronic communication (e.g., wires 80) with any number of different components therein. Any number of different end effectors (e.g., 16) may be configured to house any number of different shaker motors without departing from the scope of the present invention. The shaker motor (e.g., in end-effector 16) may provide the mechanical stimulus for implementing vibrations to the patient's arm 26. The end-effector 16 may be adjusted in any number of different directions in three dimensions, based on, by way of example and not limitation, a positioning apparatus 44 (e.g., 3-motor standard delta-robot motor configuration). Motors, computers, other electrical equipment, some combination thereof, or the like may be housed in a control cabinet, which may be positioned above a vertical post. The patient's limb to be tested, such as the patient's arm 26 may be immobilized by an adjustable wrist constraint 24 (e.g., supported by adjustable support 36) and an adjustable elbow constraint 18.

An exemplary shaker assembly may comprise bronze ballast rings, and a shaker motor suspended between springs on a top and bottom portion thereof, wherein the springs may be fixed between the ballast rings and both a bottom mounting plate and top mounting plate. Lobed tabs may be positioned on each mounting plate to act as guides to constrain the motion of the shaker assembly only to an axial direction, although such is not necessarily required. Vibration isolation material may comprise Sorbothane™, which may be derived from polyurethane. Sorbothane™ may be positioned between the shaker assembly and its housing to reduce random noise in response data. Exemplary springs may be configured to minimize random noise in response data. The aforementioned mechanization of the probe 74 may provide an exemplary ability to immobilize and adjust an arm 26 at any number of locations before and/or during data acquisition, and to reduce random noise in measured responses to mechanical vibrations.

In an exemplary embodiment, once an individual is positioned correctly with respect to the device 10 (e.g., may take 5-8 minutes), the test may be initiated by positioning the end effector apparatus 16 over a mid-point of the ulna (e.g., of arm 26). An automated CBMT instrument may then signal a delta robot (e.g., utilizing three linear actuators) to lower the probe 74 into approximate contact with the ulna bone. The end effector 16 may include a mechanical shaker, an impedance head, a ceramic saddle-shaped, patient-contact probe (e.g., 74), some combination thereof, or the like. A downward displacement of the probe 74 may apply a prescribed static load (e.g., 6 N≤ Static Load≤18 N). The shaker may then be driven by an excitation signal superimposed on the static load comprising a band-limited (e.g., 20-1600 Hz) increasing and/or decreasing repeating chirp sequence (e.g., zero mean, 6 N span) or other band-limited random white noise. The applied force and resulting acceleration of bone (e.g., of an ulna-skin complex) may be measured by an impedance head and may be sampled (e.g., at 16 kHz) by a two-channel in-line signal conditioner.

Referring now to FIGS. 34-37, another exemplary CBMT device 10B is shown having a horizontal support member 35, support apparatus 30, and vertical post 46 each providing structural support to at least one of various other components (e.g., control cabinet 14, table 34) of the device 10B. One or more support feet 142 may provide base support and floor gripping for the horizontal support member 35. One or more displays (e.g., 38, 38A-B) may be connected to the vertical post 46 by way of a mechanical connection 40. The mechanical connection 40 may be received by and secured to a groove of the vertical post 46, although such is not required. The control cabinet 14 may be connected to the vertical post 46 by way of one or more connection brackets 152 configured to be received by and secured to the vertical post 46. The aforementioned connections may be achieved by any number of different bolts, fasteners, clips, clamps, some combination thereof, or the like.

The position of the table 34 may be adjusted to, e.g., provide for variable limb positioning of any number of different patients of any number of different sizes. For example, the table 34 may be directed from a right to left position to permit bone vibration testing of a user's left arm, and vice versa. The position of the table 34 may be adjusted by way of direct adjustment of an adjustable base platform 150 below the table 34. Side-to-side movement of the adjustable base platform 150 may be manually controlled by engaging a side-to-side position adjustment knob 146 (which may be positioned at either end of the base platform 150). The table 34 may be moved forward or backward (e.g., to accommodate different patient heights and/or limb lengths) by manual engagement of a front-to-back position adjustment knob 148. Alternatively, or additionally, positioning of the adjustable base platform 150 may be controlled by one or more electronic actuators, which may be controlled remotely, although such is not required. It will be apparent to one or ordinary skill in the art that there may be any number of different techniques available for adjusting an exemplary table without departing from the scope of the present invention.

The exemplary CBMT device 10B of FIGS. 34-37 further comprises a wrist constraint 24 linked to a vertical support 36 having a vertical support adjustment knob 144, an elbow positioner 180 having padded bars 180B, and linked to a vertical frame member 20 and a horizontal frame member 23, and a shaker assembly isolation system 160 positioned below and controlled by a positioning apparatus 44. The positioning apparatus 44 may comprise a 3-motor standard delta-robot motor configuration, although such is not required. The positioning apparatus 44 may be configured to control the exact position across 3 dimensions of the shaker assembly isolation system 160 with respect to a patient positioned on the table 34. It will be apparent to one of ordinary skill in the art that any number of different techniques or devices may be employed to control positioning of an exemplary shaker assembly isolation system without departing from the scope of the present invention.

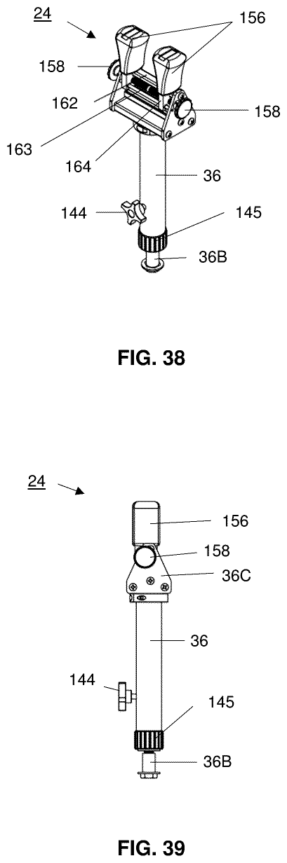

Referring now to FIGS. 38-41, the exemplary wrist constraint 24 may provide for immobilization of a human arm at or near one's wrist (e.g., to promote stabilization of the ulna). Specifically, the wrist constraint 24 may permit the patient's wrist to be centered and braced under an adjustable data collection probe. The wrist constraint 24 may include a pair of ergonomic constraining braces 156 each comprising an amount of cushioning material. One or more horizontal control knobs 158 (e.g., positioned on an upper vertical support member 36C) may permit each brace 156 to be positioned at one of an infinite number of distances from the other brace 156 to secure a wrist therebetween. The horizontal control knob may be rotated a first direction to position the braces 156 closer to one another (e.g., to secure a smaller wrist), and may be rotated a second direction opposite of the first direction to position the braces 156 farther from one another (e.g., to secure a larger wrist).

Each brace 156 may be associated with a control knob 158 specifically configured to permit position control of the brace 156. Rotation of the control knob 158 may cause an articulating paddle 164 affixed to the brace 156 to move horizontally along a threaded leadscrew 163, thus causing horizontal repositioning of the brace 156. The threaded leadscrew 163 may be secured below a wrist support platform 162, the wrist support platform 162 configured to support and promote immobilization of the wrist. The threaded leadscrew 163 may comprise left and/or right threads opposing corresponding threads of the articulating paddle 164. Rotation of the control knob 158 may cause rotation of the threaded leadscrew 163, thus causing movement of the articulating paddle 164. Each the constraining braces 156 and the wrist support platform 162 may contact the upper portion of one's wrist when one's arm is positioned for vibration testing. The wrist may be immobilized to achieve ulna alignment parallel to the table and perpendicular to a testing probe, wherein the testing probe is adapted to be positioned at a midpoint of the ulna.

The height of the wrist support platform 162 and constraining braces 156 may be adjusted by adjusting the length of a vertical support member 36. In the embodiment shown, a vertical support adjustment knob 145 may be rotated in a first direction to cause a main portion of the vertical support member 36 to move upwards from a lower vertical support member 36B, and may be rotated in a second direction to cause the main portion of the vertical support member 36 to move downwards over the lower vertical support member 36B. Height adjustment of the wrist support platform 162 and constraining braces 156 may also be achieved by direct or remote control of an electronic actuator adapted to cause upward/downward movement of a main portion of a vertical support member. When an optimal height position is obtained, the vertical support member 36 may be locked into place with a locking knob 144. It will be apparent to one of ordinary skill in the art that there may be any number of different methods for regulating the height of a wrist constraint 24 without departing from the scope of the present invention.

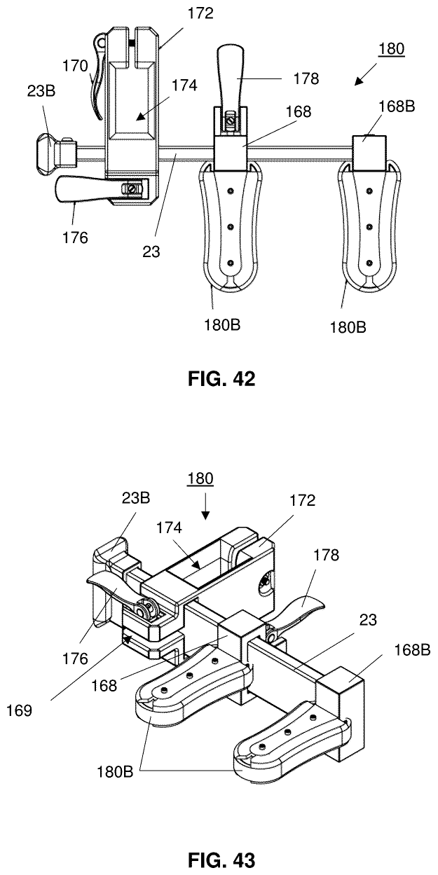

Referring now to FIGS. 42-44, the exemplary elbow positioner 180 may include a clamping mechanism with several degrees of motion for immobilizing an arm at or near the elbow. Cushioning material of padded bars 180B of the elbow positioner 180 may promote user comfort. The elbow positioner 180 may be configured to promote accurate and repeatable desired positioning of an elbow, such as to cause the proximal side of the forearm to be isolated and immobilized proximate to a data testing probe. A patient's elbow may be secured between the padded bars 180B. A lockable bearing block 172 may be configured to slide along a vertical frame member (20 in FIG. 34) to permit height adjustment of the elbow positioner 180. A pair of horizontal sliding joints 168, 169 may be configured to slide along a horizontal frame member 23 to permit horizontal adjustment of the elbow positioner 180.

In this particular embodiment, a first horizontal sliding joint 168 permits horizontal adjustment of a first padded bar 180B, and a second horizontal sliding joint 169 permits horizontal adjustment of the lockable bearing block 172. The first horizontal sliding joint 168 may permit the first padded bar 180B to be positioned against the medial side of a patient's forearm, under the epicondyles of the humerus. A cam actuated lever 178 may be engaged to permit the sliding joint 168 to slide along the horizontal frame member 23 (e.g., to permit the first padded bar 180B to be positioned firmly against the patient's forearm). A second padded bar 180B may be connected to a joint 168B which may be horizontally adjustable, although such is not required. The second padded bar 180B may be located on the lateral side of a patient's arm, under the epicondyles of the humerus. Either padded bar 180B may be repositioned along the horizontal frame member 23 to make parallel contact with a patient's arm opposite of the other padded bar 180B to immobilize the arm proximate to the elbow. Either joint 168, 168B connected to a padded bar 180B may comprise a lockable bearing block, which may be unlocked using a cam actuated lever (e.g., 178). When the cam actuated lever 178 is not engaged, a protrusion (not shown) of the joint (e.g., 168) may secure the joint to the horizontal frame member 23.

A horizontal frame member guard 23B may be positioned on an end of the horizontal frame member 23 opposite of the joint 168B to, e.g., prevent the lockable bearing block 172 from becoming disengaged from the horizontal frame member 23. The horizontal frame member guard 23B may also provide for user gripping for repositioning the horizontal frame member 23. The lockable bearing block 172 may slide along the horizontal frame member 23 at the horizontal sliding joint 169, which may be specifically sized to receive the horizontal frame member 23. In an exemplary embodiment, where the horizontal frame member 23 is mobilized, the medially located padded bar 180B is adapted to be in a fixed position. In the aforementioned embodiment, where the horizontal frame member 23 is in a fixed position, the medially located padded bar 180B is permitted to slide horizontally along the horizontal frame member 23.

A vertical member receptacle 174 of the bearing block 172 may be configured to receive the vertical frame member (20 of FIG. 34). A cam actuated lever 176 positioned on the lockable bearing block 172 opposite of the vertical member receptacle 174 may be engaged to unlock the bearing block 172 to permit the bearing block 172 to be repositioned along the horizontal frame member 23 (e.g., to permit repositioning of the horizontal frame member 23 with respect to the vertical frame member). For example, horizontal repositioning of the horizontal frame member in either the left or right direction may permit positioning the ulna of the forearm parallel to the long edge of the table (34 in FIG. 34). When the cam actuated lever 176 is not engaged, a protrusion 182 of the block 172 may secure the block 172 to the horizontal frame member 23.

Another cam actuated lever (e.g., 170) positioned on the lockable bearing block 172 adjacent to the vertical member receptacle 174 may be engaged to unlock the bearing block 172 to permit the bearing block 172 to be repositioned along the vertical frame member (e.g., to permit height adjustment of the elbow positioner 180). For example, the cam actuated lever 170 may be engaged to permit the horizontal frame member 23 to be raised to cause the patient's arm to be lifted up vertically (e.g., by way of pressure applied to the proximal side of the epicondyles). When the cam actuated lever 170 is not engaged, a protrusion (not shown) of the block 172 may secure the block 172 to the vertical frame member (20 in FIG. 34). The cam actuated lever controlling repositioning of the bearing block along the vertical frame member may alternatively or additionally be positioned opposite of the vertical member receptacle 174. It will be apparent to one of ordinary skill in the art that the particular position(s) of cam actuated levers illustrated and described herein may be varied without departing from the scope of the present invention.

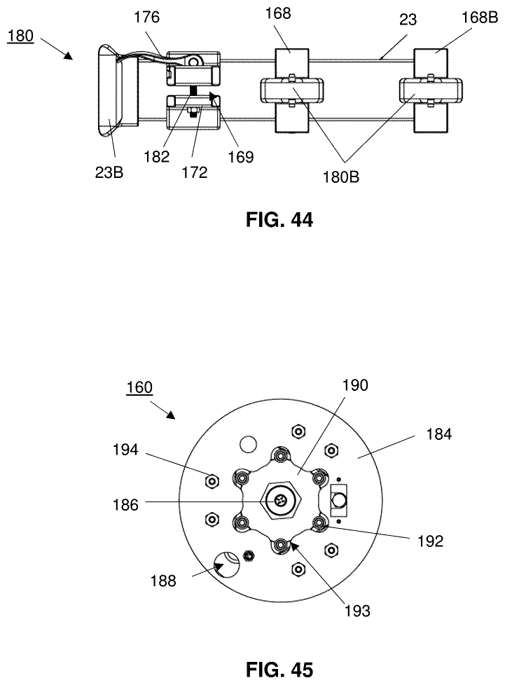

Referring to FIGS. 34 and 44, the vertical frame member 20 may be affixed to the frame of the table 34. Vertical frame members 20 may be positioned on either side of the table 34 to permit limbs on either side of the patient's body to be tested. The horizontal frame member 23 may be disengaged from the vertical frame member 20 and rotated 180 degrees to vary the direction the padded bars 180B are facing. The application of pressure applied by the padded bars 180B to the patient's forearm on the humerus below the epicondyles may cause the forearm to be isolated and immobilized with respect to the rest of the patient's body. It will be apparent to one of ordinary skill in the art that the elbow positioners described herein are merely illustrative, and variations to an exemplary limb positioner may be made without departing from the scope of the present invention.

Referring now to FIGS. 45-52, an exemplary shaker assembly isolation system 160 may surround a shaker assembly 190, and positioning of the shaker assembly 190 may be regulated by one or more linear guide bearings 200. The shaker assembly isolation system 160 may also comprise a top plate 184 and bottom plate 198, wherein at least one vertical frame member 196 may be secured to the top plate 184 by one or more fasteners 194, and to the bottom plate 196 by one or more fasteners 194B. The shaker assembly 190 may be centrally positioned within the isolation system 160. The shaker assembly 190 may comprise a linear shaker motor 202 which may be configured to direct vibration stimuli at or proximate to a midpoint of an ulna. A data collection probe 208 may be positioned between the linear shaker motor 202 and a midpoint of a forearm. The data collection probe 208 may be affixed to a support member 206 protruding below the shaker assembly 190 and in mechanical communication with the linear shaker motor 202.

The shaker assembly 190 may be contained within the isolation system 160 by vertical frame members 196 and linear guide bearings 200 received by the vertical frame members 196. Specifically, each linear guide bearing 200 may comprise a front member 218, a pair of intermediate members 222, and a rear member 224. The front member 218 may include a pair of t-slot mating features 216 adapted to be received by the vertical frame members 196. The intermediate members 222 may comprise moldable polymeric material adapted to be received in receptacles 220 of the front member 218 to link the front member 218 and the rear member 224, and reduce vibration propagation therebetween. The rear member 224 may comprise a shaker assembly mating feature 226 defined by a curved surface having a pair of mating channels 228 each configured to receive a column 209 of the shaker assembly 190.

The shaker assembly 190 may comprise a roof portion 210 and a base portion 204, wherein columns 209 may extend the distance from the roof portion 210 to the base portion 204. Each column 209 may comprise a fastener (e.g., a bolt). The roof portion 210 and/or the base portion 204 may include an amount of moldable polymeric material 193. In this particular embodiment, a bolt head 192 and washer secure each column 209 to the roof portion 210, and a nut 192B having threads configured to mate with corresponding threads of the fastener secures each column 209 to the base portion 204. An axial fastener 186 may further secure various electrical connection components of the shaker assembly 190 between the roof portion 210 and the base portion 204. One or more apertures (e.g., 188) may be provided on the top plate 184 of the shaker assembly isolation system 160 to permit access to various components of the system 160.

The isolation system 160 may be configured to dampen transmission of spurious vibrations from the shaker assembly 190 such that measurements of the data collection probe are isolated to measurements of motion of tissue (e.g., motion of the skin and bone complex in the forearm). Various components of the isolation system 160 and/or shaker assembly 190 may include materials adapted to reduce spurious vibrations, including by way of example and not limitation, moldable polymeric material (e.g., polyurethane). For example, vibrations propagating through support structures such as the vertical frame members with t-slots 196 and linear guide bearings 200 may be absorbed by said materials to prevent random noise in collected data.

As a specific, non-limiting example, where the shaker assembly 190 is positioned above the bottom plate 198 of isolation system 160, moldable polymeric material 193 may be applied between the base 204 of the shaker assembly 190 and the bottom plate 198 of the isolation system 160. Pressure sensitive adhesive may be used to secure the moldable polymeric material 193 to the top surface of the bottom plate 198 and/or the bottom surface of the base 204 of the shaker assembly 190. A thin film of polyethylene and/or other similar material may be applied to either side of the moldable polymeric material 193 to prevent the top surface of the bottom plate 198 and the bottom surface of the base 204 from sticking to one another. The positioning of said material within system 160 may isolate the support structure (e.g., vertical frame members with t-slots 196) of system 160 from axial vibrations of the shaker assembly 190.

Said materials may be formulated with varying densities, material hardness (e.g., measurable using a durometer), and the like. The magnitude of loads applied and material densities used in the system 160 may influence vibration isolation characteristics, thus careful matching of an appropriate polymeric material density to a particular applied load may result in an optimized damping response. The system 160 may be configured to travel throughout 3 dimensions (e.g., using a positioning apparatus) to optimize the location of the test stimulus application (e.g., on a patient's forearm).

The linear guide bearings 200, connected to the vertical frame members with t-slots 196, confine movement of the shaker assembly 190 to axial directions. The t-slots of vertical frame members 196 may receive t-slot mating features 216 of the linear guide bearings 200 to permit free vertical motion of the shaker assembly 190 within the confines of the isolation system 160, but restrict horizontal motion thereof. The t-slot mating features 216 may be permitted to slide upward along t-slots of vertical frame members 196, e.g., as a safety mechanism to prevent overloading of force on a limb during vibration application.

In the particular embodiment shown, three linear guide bearings 200 of isolation system 160 are positioned between top 184 and bottom 198 plates, and are each adapted to receive a pair of shaker assembly columns 209 (which may each clip into a mating channel 228). The intermediate members 222 of each linear guide bearing 200, which may comprise moldable polymeric material, and which may mate the front member 218 to the rear member 224, may cause the shaker assembly 190 to be radially isolated from the front member 218 and vertical frame member 196. Said radial isolation may prevent vibrations of the shaker assembly 190 from propagating into support structures of the isolation system 160.

The shaker assembly 190 may be precisely ballasted such that its weight is greater than any combination of axial vibratory and static loads applied by the motor 202 or data collection probe 208 (e.g., to prevent the shaker assembly 190 from lifting off of the bottom plate 198). Upper 212A and lower 212B ballast rings may be secured between the roof 210 and base 204 of the shaker assembly to provide the ballast. The ballast rings 212A, 212B may also define housing of the motor 202. It will be apparent to one of ordinary skill in the art that the particular shaker motor and isolation system configurations illustrated and described herein are in no way exhaustive of the scope of the present invention, and variations to said configurations may be made without departing from the scope of the present invention.

Referring now to FIGS. 9-10, an exemplary modal transmission model 82 and related equation 84 are shown. The model 82 may account for the mass of the skin (Mₛ), transverse bending stiffness of the skin (Kₛ), damping coefficient of the skin (Cₛ), mass of the bone (M_{b}), transverse bending stiffness of the bone (K_{b}), damping coefficient of the bone (C_{b}), and damping of peripheral flesh (Cₚ). As expressed by exemplary equation 84, which demonstrates a relationship between (1) displacement of the bone divided by force applied thereto, as a function of angular frequency, and (2) modal amplitude factors, damping ratios, and resonant frequencies of skin and bone, once certain parameters are determined, other parameters may be calculated for by an exemplary system.

Work in the model space regarding sFRF or cFRF data and solutions to the 7-parameters may be estimated based off of seed values provided to the fitting routine (e.g., providing a least-squares error fitting algorithm, as described above). Solutions may be stored to a disk or other digital storage medium. As a measure of conformity, the mean-squared error (MSE) term calculated and returned by the fitting routine may be compared for all data sets, and parameters associated with a data set having minimized MSE may be selected as an optimal set. An exemplary windowing function may permit model parameters to be fitted to the sFRF or cFRF directly. By way of example and not limitation, transverse bending stiffness of the bone (K_{b}) may be determined based on estimation according to seed values provided to the fitting routine. Since bone stiffness may correlate closely with bone strength and fracture potential, said (K_{b}) determination of a particular patient may assist a doctor thereof in evaluating the patient's bone strength and fracture potential.

Referring now to FIGS. 11-18, exemplary graphs 86, 90, 94, 96, 98, 100, 102, 104 are shown, each representing various functions of an exemplary system. Exemplary graph 86 represents an exemplary force (e.g., applied by probe) versus displacement curve in accordance with an exemplary embodiment. Exemplary graph 90 also represents Force (N) compared to displacement (mm) by an exemplary system. Exemplary graphs 94 and 96 represent both real and imaginary accelerance curves in accordance with an exemplary embodiment. It will be apparent to one of ordinary skill in the art that representing accelerance data as real and imaginary data is not necessarily required. Complex notation may also be used to express magnitude and phase information of accelerance compared to frequency.

Exemplary graph 98 represents a complex compliance FRF (grey line) and best model fit (black line), each demonstrating resonances at approximately 200 and 800 Hz. The location and shape of the higher frequency resonance may be determined primarily by mechanical properties of the skin and the applied static load (referred to herein as “soft tissue peak”). The location and shape of the lower frequency resonance may be determined primarily by mechanical properties of the underlying bone (referred to herein as “bone peak”). Both resonances may also be affected by damping effects of surrounding soft tissue. By way of example and not limitation, non-biological low frequency (<50 Hz) noise may be attributed to the mechanical system. An exemplary model may be configured to fit data between 70-1,600 Hz. Thus, an exemplary model is not necessarily influenced by the aforementioned low frequency noise. Ulnar flexural rigidity may be ultimately quantified based on a compliance FRF model fit. Exemplary graph 100 represents both real and imaginary curves expressing compliance versus frequency in accordance with an exemplary embodiment, and exemplary graph 102 alternatively represents complex notation for expressing stiffness versus frequency. Exemplary graph 104 represents complex notation of compliance for expressing an optimized model fit to data. It will be apparent to one of ordinary skill in the art that there may be any number of different methods for organizing and displaying exemplary data without necessarily departing from the scope of the present invention.

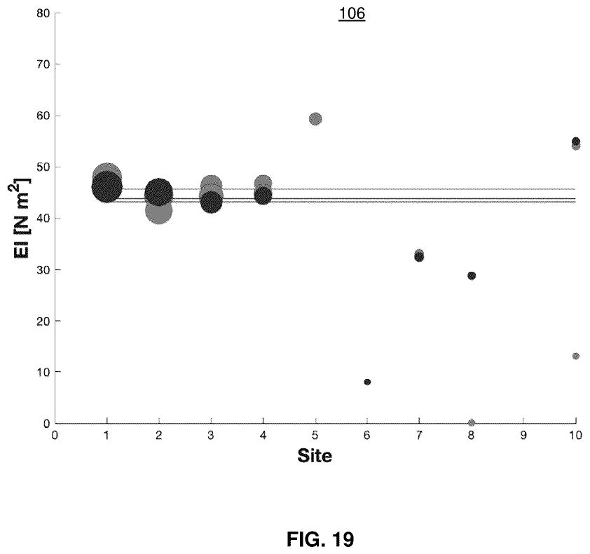

Referring now to FIGS. 19-30, exemplary graphs 106, 112, 118 and 124 demonstrate a number of instances of flexural rigidity (N m²) of bone calculated for various locations across the width of a bone. Flexural rigidity for each of various sites may be computed as a function of bone stiffness and length in accordance with known methods. Exemplary graphs 108, 114, 120 and 126 demonstrate a number of instances of compliance (x/F) magnitude being plotted versus mechanical vibration frequency for a first bone site. Exemplary graphs 110, 116, 122, and 128 demonstrate a number of instances of compliance (x/F) magnitude being plotted versus mechanical vibration frequency for a second bone site.

Referring now to FIG. 31, an exemplary purpose for assessing ulnar flexural rigidity at a mid-point 132 of an ulna 130, where a CBMT probe may be positioned, is that the ulna 130 substantially comprises cortical bone at the mid-point 132. Cortical bone loss may be more associated with increased incidence of fragility fractures. Cortical bone loss is also particularly prevalent in individuals between 65 and 79 years of age (e.g., demonstrated by graph 134), especially when compared to trabecular bone loss. In individuals over 80 years of age, the vast majority of bone loss may be attributed to cortical bone loss (see e.g., graph 136). It will be apparent to one of ordinary skill in the art that there may be any number of different advantages to assessing cortical bone loss using an exemplary CBMT device.

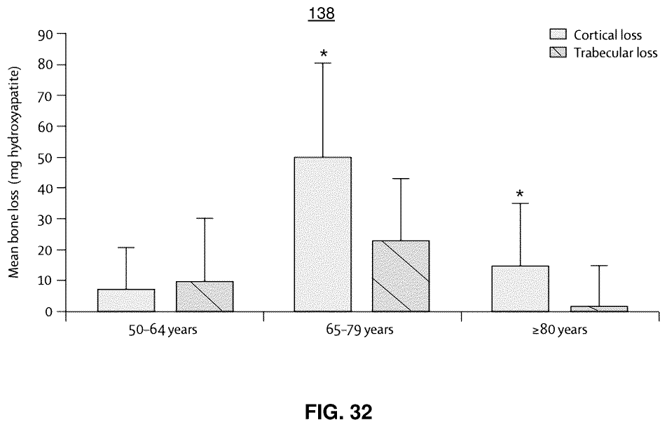

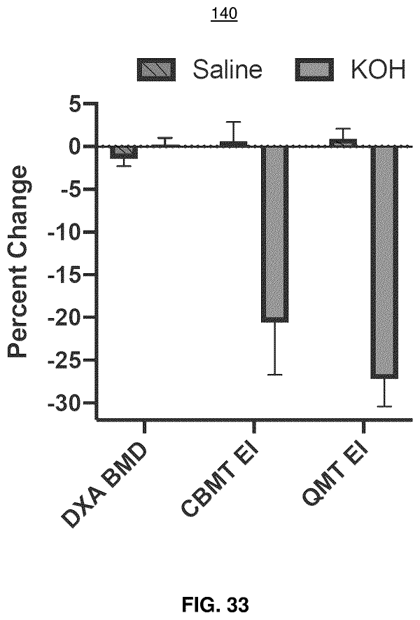

FIG. 32 is another exemplary graph 138 demonstrating that cortical bone loss is the predominant form of bone loss in elderly individuals. FIG. 33 is yet another exemplary graph 140, this particular graph 140 demonstrating the effects of 39-hours of saline or potassium hydroxide on DXA-derived measures of bone mineral density (DXA BMD), and flexural rigidity (EI), as measured by CBMT and quasistatic mechanical testing (QMT). It will be apparent to one of ordinary skill in the art that any of the aforementioned graphs may be displayed by any number of different interfaces without departing from the scope of the present invention. It will also be apparent to one of ordinary skill in the art that the aforementioned graphs are merely illustrative, and are in no way meant to be exhaustive of the scope of the present invention.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A system for evaluating mechanical properties of bone tissue, the system comprising:
a limb support comprising a first, adjustable constraint for constraining a first location on said limb, and a second, adjustable constraint for constraining a second location on said limb of a test subject;
a probe, configured to apply static and oscillatory forces over a range of frequencies to said limb;
a shaker assembly, positioned in a shaker assembly isolation system;
wherein said probe is in mechanical communication with said shaker assembly; and
wherein said shaker assembly isolation system is configured to dampen transmission of vibrations from said shaker assembly.

2. The system of claim 1 further comprising:
a processor, configured to receive vibration response data from said limb and analyze said data to provide an output corresponding to bone transverse bending stiffness.

3. The system of claim 1 wherein:
the second constraint comprises a pair of padded bars configured to secure an elbow therebetween.

4. The system of claim 1 wherein:
the first constraint comprises first and second clamps, the first clamp configured to be positioned proximate to the second clamp to secure a wrist of the test subject therebetween.

5. The system of claim 1 wherein:
the shaker assembly isolation system comprises one or more linear guide bearings.

6. The system of claim 1 further comprising:
a beam adjustable along a support member, and a bearing block and sliding joint connected to the beam, each adjustable to form said second constraint.

7. The system of claim 1 wherein:
the shaker assembly and shaker assembly isolation system are connected to a positioning apparatus capable of moving the shaker assembly and shaker assembly isolation system throughout 3 dimensions.

8. The system of claim 4 further comprising:
a threaded leadscrew, positioned below said first and second clamps, and configured to permit a distance between said first and second clamps to be varied.

9. The system of claim 5 wherein:
an amount of moldable polymeric material is positioned below the shaker assembly.

10. The system of claim 1 wherein:
the probe is capable of applying static and oscillatory forces over a range of frequencies to the test subject at a number of different locations proximate to an ulna.

11. The system of claim 6 further comprising:
a cam lever, configured to permit regulation of movement of said bearing block.

12. A system for evaluating mechanical properties of bone tissue, the system comprising:
an arm support comprising an adjustable wrist constraint and an adjustable elbow constraint for immobilizing a limb of a test subject;
a probe, configured to apply static and oscillatory forces over a range of frequencies to said limb;
a shaker assembly isolation system, connected to a positioning apparatus, and comprising a top plate, bottom plate, and one or more linear guide bearings positioned between said top plate and bottom plate; and
a shaker assembly, positioned between said top plate and bottom plate, and adjacent to said one or more linear guide bearings;
wherein said probe is in mechanical communication with said shaker assembly; and
wherein said shaker assembly isolation system is configured to dampen transmission of vibrations from said shaker assembly.

13. The system of claim 12 further comprising:
a processor, configured to receive vibration response data from said limb and analyze said data to provide an output corresponding to bone transverse bending stiffness.

14. The system of claim 12 wherein:
an amount of material adapted to reduce transmission of vibrations between said shaker assembly and said shaker assembly isolation system is positioned on at least one selected from the group of said one or more linear guide bearings, said top plate, and said bottom plate.

15. The system of claim 14 wherein said material is a moldable polymeric material.

16. The system of claim 12 wherein:
the adjustable elbow constraint comprises a first clamp configured to be positioned proximate to a second clamp to secure an elbow proximate thereto, each the first clamp and the second clamp movable along and adapted to be secured to a horizontal support member; and
the adjustable wrist constraint comprises a first clamp configured to be positioned proximate to a second clamp to secure a wrist therebetween.

17. A method for evaluating mechanical properties of bone tissue, the method comprising:
providing an arm support comprising an adjustable wrist constraint and an adjustable elbow constraint for immobilizing a limb of a test subject;
providing a probe, configured to apply static and oscillatory forces over a range of frequencies to the test subject;
providing a shaker assembly, positioned in a shaker assembly isolation system, wherein the shaker assembly is in mechanical communication with the probe;
configuring the adjustable elbow constraint to further include a first clamp adapted to be positioned proximate to a second clamp to secure an elbow proximate thereto, wherein at least one selected from the group of the first and second clamp is movable along and adapted to be secured to a horizontal support member; and
configuring said shaker assembly isolation system to dampen transmission of vibrations from said shaker assembly.

18. The method of claim 17 further comprising:
configuring the adjustable wrist constraint to further include a first clamp, and configuring the first clamp to be positioned proximate to a second clamp to secure a wrist therebetween.

19. The method of claim 17 further comprising:
providing a processor, configured to receive vibration response data from said limb and analyze said data to provide an output corresponding to bone transverse bending stiffness.

20. The method of claim 17 further comprising:
providing a linear shaker motor surrounded by at least one ballast ring to define a portion of the shaker assembly.
